(19) United States
(12) Patent Application Publication
(10) Pub. No.: US 2018/0357375 A1
(43) Pub. Date: Dec. 13, 2018

(54) METHODS AND COMPOSITIONS FOR DETERMINING METABOLIC MAPS

(71) Applicant: Whole Biome Inc., San Francisco, CA (US)

(72) Inventors: Colleen CUTCLIFFE, Menlo Park, CA (US); John S. EID, San Francisco, CA (US); James H. BULLARD, San Francisco, CA (US); Tomer ALTMAN, San Francisco, CA (US); Fanny PERRAUDEAU, San Francisco, CA (US)

(21) Appl. No.: 15/945,705

(22) Filed: Apr. 4, 2018

Related U.S. Application Data
(60) Provisional application No. 62/481,654, filed on Apr. 4, 2017.

(51) Int. Cl. G06F 19/26; G06F 19/28; G06F 19/24; G06F 19/20; C12Q 1/6874

(52) U.S. Cl. CPC G06F 19/26; G06F 19/28; C12Q 1/6874; G06F 19/20; G06F 19/24

(57) ABSTRACT

The present disclosure provides methods of determining metabolic maps and identifying presence of and estimating abundances of microbiome metabolic pathways in an individual toward customized microbial therapy. In an aspect, the present disclosure provides a method of determining an abundance of a metabolic pathway from a sample comprising a population of a plurality of different organisms.

This application claims priority to U.S. Provisional Application No. 62/481,654, filed Apr. 4, 2017, which is incorporated herein by reference in its entirety for all purposes.

### STATEMENT AS TO FEDERALLY SPONSORED RESEARCH

This invention was made with government support under 1R41GM121144-01 awarded by National Institute of General Medical Sciences, NIH.

### BACKGROUND OF THE DISCLOSURE

The microbiome can play an important role in maintaining physiological functions of the body. Dysbiosis of the microbiome can lead to various disorders. Clinical phenotypes like obesity, inflammatory bowel disease, type 1, and type 2 diabetes, and various mental states can be linked with molecular signatures in the human microbiome. Determination of microbial pathways and their signatures across a variety of diseases can aid identification of relevant therapeutic interventions.

### SUMMARY OF THE DISCLOSURE

In one aspect, the present disclosure provides a method of determining an abundance of a metabolic pathway from a sample comprising a population of a plurality of different organisms, the method comprising: (a) obtaining sequencing information from nucleic acid molecules in the population; (b) determining a presence of a nucleic acid marker that encodes a component of the metabolic pathway in a genome of each of one or more organisms in the plurality of different organisms in the population, comprising: (i) identifying an organism in the population based on the sequencing information, (ii) for the organism of (i), identifying a set of reactions, and (iii) determining a presence of the nucleic acid marker from the organism in the identified set of reactions, wherein the nucleic acid marker encodes the component of the metabolic pathway in the genome of the organism; and (c) determining the abundance of the nucleic acid marker from the plurality of different organisms in the population, thereby determining an abundance of the metabolic pathway in the population.

In some embodiments, wherein the organism is a microbe, and wherein the population comprises a population of microbes. In some embodiments, the abundance comprises a relative abundance. In some embodiments, the abundance comprises a normalized abundance. In some embodiments, the nucleic acid marker encodes an enzyme in the metabolic pathway. In some embodiments, (c) comprises determining the abundance of the metabolic pathway based at least in part on an abundance of the nucleic acid marker that comprises a sequence encoding an enzyme in the metabolic pathway. In some embodiments, the metabolic pathway comprises a distributed metabolic pathway. In some embodiments, the distributed metabolic pathway is catalyzed by a plurality of organisms. In some embodiments, the distributed metabolic pathway comprises a microbiome distributed metabolic pathway. In some embodiments, the microbiome distributed metabolic pathway is associated with a plurality of microbes.

In some embodiments, determining the presence of the metabolic pathway comprises querying a database based on the organism identified in (i). In some embodiments, the metabolic pathway is identified using a model trained with metabolic pathway data that is tiered, each tier of metabolic pathway data corresponding to a different discrete range of confidence level in the metabolic pathway data. In some embodiments, the method further comprises generating one or more feature vectors for the organism and the nucleic acid marker from the organism. In some embodiments, the one or more feature vectors are selected from the group consisting of: reaction-info-content-norm, fraction-reactions-with-enzymes, taxonomic-range-includes-target-alt, enzyme-info-content-norm, all-rxns-are-present, num-pathway-holes, not-mostly-absent, rxn-set-difference, manually-curated-parts, partial-pwy-evidence, manually-curated, glycan-pathway, and any combination thereof. In some embodiments, the method further comprises using the one or more feature vectors to determine the abundance of the nucleic acid marker from the organism, wherein the one or more feature vectors are indicative of presence of the metabolic pathway. In some embodiments, determining the presence of the nucleic acid marker from the organism further comprises using a machine learning algorithm trained with a set of metabolic pathways that are known to be present or absent in the one or more organisms in the plurality. In some embodiments, the machine learning algorithm is configured to determine the abundance nucleic acid marker of a distributed metabolic pathway. In some embodiments, the distributed metabolic pathway is catalyzed at least in part by two or more microbes in the population. In some embodiments, the distributed metabolic pathway has transporters for intermediate metabolites catalyzed by the microbe. In some embodiments, the machine learning algorithm comprises a random forest. In some embodiments, the sample comprises an environmental sample. In some embodiments, the sample comprises a biological sample. In some embodiments, the biological sample comprises fecal matter. In some embodiments, the metabolic pathway is associated with production of short-chain fatty acids (SCFAs). In some embodiments, obtaining the sequencing information comprises sequencing a nucleic acid sequence of a ribosomal RNA operon in the sample.

In some embodiments, the presence of the nucleic acid marker is identified with a mean or median accuracy of at least about 92%. In some embodiments, the presence of the nucleic acid marker is identified with a mean or median accuracy of at least about 95%. In some embodiments, the presence of the nucleic acid marker is identified with a mean or median accuracy of at least about 98%. In some embodiments, the presence of the nucleic acid marker is identified with a mean or median accuracy of at least about 99%. In some embodiments, the presence of the nucleic acid marker is identified with a mean or median accuracy of at least about 99.5%. In some embodiments, the presence of the nucleic acid marker is identified with a mean or median sensitivity of at least about 80%. In some embodiments, the presence of the nucleic acid marker is identified with a mean or median specificity of at least about 96%. In some embodiments, the presence of the nucleic acid marker is identified with a mean or median specificity of at least about 99%.

In another aspect, the present disclosure provides a method for estimating changes in a metabolic pathway in an organism in a population of organisms in a first sample obtained at a first time and a second sample obtained at a second time, comprising: (a) obtaining a first set of sequencing information from the first sample obtained at the first time and a second set of sequencing information from the second sample obtained at a second time, each of the first sample and the second sample comprising an organism, wherein the first set of sequencing information and the second set of sequencing information comprise a nucleic acid marker from the organism, wherein the nucleic acid marker encodes a component of the metabolic pathway in a genome of the organism; (b) for the first sample and the second sample, determining an abundance of the metabolic pathway for the organism based the abundance of the nucleic acid marker from the organism; (c) performing a time series analysis from the first time to the second time for the organism; and (d) generating a metabolic profile for the first sample and the second sample using the time series analysis.

In some embodiments, the abundance comprises a relative abundance. In some embodiments, the abundance comprises a normalized abundance. In some embodiments, the nucleic acid marker encodes an enzyme in the metabolic pathway. In some embodiments, (b) comprises determining the abundance of the metabolic pathway based at least in part on an abundance of nucleic acid marker encoding an enzyme in the metabolic pathway. In some embodiments, the metabolic pathway comprises a distributed metabolic pathway. In some embodiments, the distributed metabolic pathway is associated with a plurality of microbes. In some embodiments, the distributed metabolic pathway comprises a microbiome distributed metabolic pathway. In some embodiments, the distributed metabolic pathway is associated with a plurality of microbes. In some embodiments, the first sample and the second sample are biological samples from a subject. In some embodiments, the biological samples comprise fecal matter. In some embodiments, the subject is a human. In some embodiments, the metabolic pathway is associated with production of short-chain fatty acids (SCFAs). In some embodiments, the SCFAs comprise butyrate. In some embodiments, the method further comprises administering a composition comprising one or more microbes to the subject based at least in part on the metabolic profile. In some embodiments, the composition comprises butyrate-producing microbes. In some embodiments, the first sample and the second sample are collected from the same source. In some embodiments, the first sample and the second sample are collected from different sources. In some embodiments, the time series analysis is performed using an analysis selected from the group consisting of: time-decay, detrending, augmented-Dickey Fuller test, cross-correlation, similarity analysis (LSA), time-varying network inference, auto-correlation, auto-correlogram, Hurst exponent, Lyapunov exponent, predictability analysis, bistability analysis, early warning signs, and a combination thereof.

In another aspect, the present disclosure provides a method for detecting a population of microbes in a subject, which population of microbes is responsive to administration of a composition comprising one or more butyrate-producing microbes, the method comprising: (a) obtaining a biological sample from the subject comprising a population of microbes and one or more metabolites; (b) assaying the biological sample to identify one or more microbes in the population and the one or more metabolites; (c) generating one or more metabolic maps based on the identified one or more microbes and the identified one or more metabolites; and (d) detecting, based on the one or more metabolic maps, the population of microbes responsive to the administration of the composition comprising the one or more butyrate-producing microbes. In some embodiments, the method further comprises administering the composition comprising the one or more butyrate-producing microbes to the subject based at least on the one or more metabolic maps. In some embodiments, the biological sample comprises fecal matter. In some embodiments, the one or more metabolic maps comprise normal butyrate levels in a sample and strains of microbes producing carbohydrates and sugars to feed the butyrate-producing strains of microbes. In some embodiments, the one or more metabolic maps are indicative of the subject being healthy. In some embodiments, the one or more metabolic maps comprise normal butyrate levels in a sample and reduced strains of microbes producing carbohydrates and sugars to feed the butyrate-producing strains of microbes. In some embodiments, the one or more metabolic maps indicate responsiveness of the subject to compositions comprising fiber. In some embodiments, the one or more metabolic maps comprise low butyrate levels in a sample and reduced strains of microbes producing carbohydrates and sugars to feed the butyrate-producing strains of microbes. In some embodiments, the one or more metabolic maps indicate a likely responsiveness of the subject to compositions comprising fiber and butyrate-producing microbes. In some embodiments, the one or more metabolic maps comprise low butyrate levels in a sample and normal strains of microbes producing carbohydrates and sugars to feed the butyrate-producing strains of microbes. In some embodiments, the one or more metabolic maps indicate a likely responsiveness of the subject to compositions comprising butyrate-producing microbes.

In some embodiments, the composition comprises one or more microbial species selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum, Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, and any combination thereof. In some embodiments, the composition comprises comprises one or more microbial selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, Faecalibacterium prausnitzii, and any combination thereof. In some embodiments, the composition comprises one or more microbial selected from the group consisting of: Akkermansia muciniphila, Clostridium beijerinckii, Clostridium butyricum, Eubacterium hallii, and any combination thereof.

In another aspect, the present disclosure provides a system, comprising: (a) a communication interface that receives, over a communication network, sequencing information generated by a nucleic acid sequencer; and (b) a computer in communication with the communication interface, wherein the computer comprises one or more computer processors and a computer readable medium comprising machine-executable code that, upon execution by the one or more computer processors, implements a method comprising: (i) receiving, over the communication network, the sequencing information of nucleic acid molecules from a population of a plurality of different organisms, (ii) detecting a presence of a nucleic acid marker that encodes a component of the metabolic pathway in a genome of each of one or more organisms in the plurality of different organisms in the population, comprising, (1) identifying an organism in the population based on the sequencing information, (2) for the organism of (1), identifying a set of reactions, and (3) determining a presence of the nucleic acid marker from the organism in the identified set of reactions, wherein the nucleic acid marker encodes the component of the metabolic pathway in the genome of the organism; and (iii) determining the abundance of the nucleic acid marker from the plurality of different organisms in the population, thereby determining an abundance of the metabolic pathway in the population.

In some embodiments, the method further comprises generating an output comprising the abundance of the metabolic pathway in the population.

In another aspect, the present disclosure provides a non-transitory computer-readable medium comprising machine executable code that, upon execution by one or more computer processors, implements a method for determining an abundance of a metabolic pathway in a population comprising a plurality of different organisms, the method comprising: (a) obtaining sequencing information for nucleic acid molecules in the population; (b) determining a presence of a nucleic acid marker that encodes a component of the metabolic pathway in a genome of each of one or more organisms in the plurality of different organisms in the population, comprising: (i) identifying an organism in the population based on the sequencing information, (ii) for the organism of (i), identifying a set of reactions, and (iii) determining a presence of the nucleic acid marker from the organism in the identified set of reactions, wherein the nucleic acid marker encodes the component of the metabolic pathway in the genome of the organism; and (c) determining the abundance of the nucleic acid marker from the plurality of different organisms in the population, thereby determining an abundance of the metabolic pathway in the population.

In another aspect, the present disclosure provides a method of determining an abundance of a metabolic pathway from a sample comprising a population of two or more different types of organisms, the method comprising: (a) obtaining sequencing information of nucleic acid molecules from the population; (b) identifying a type of organism in the population based on the sequencing information; (c) determining a presence of the nucleic acid marker from the type of organism, wherein the nucleic acid marker encodes a component of the metabolic pathway in a genome of the organism; and (d) determining the abundance of the nucleic acid marker from the type of organism, thereby determining an abundance of the metabolic pathway in the population.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The content of the International Nucleotide Sequence Database Collaboration (DDBJ/EMBL/GENBANK) accession number CP001071.1 for microbial strain Akkermansia muciniphila, culture collection ATCC BAA-835, is herein incorporated by reference in its entirety.

The content of DDBJ/EMBL/GENBANK accession number AJ518871.2 for microbial strain Anaerofustis stercorihominis, culture collection DSM 17244, is herein incorporated by reference in its entirety.

The content of DDBJ/EMBL/GENBANK accession number DS499744.1 for microbial strain Anaerostipes caccae, culture collection DSM 14662, is herein incorporated by reference in its entirety.

The content of DDBJ/EMBL/GENBANK accession number AJ270487.2 for microbial strain Anaerostipes caccae, butyrate-producing bacterium L1-92, is herein incorporated by reference in its entirety.

The content of DDBJ/EMBL/GENBANK accession number AY305319.1 for microbial strain Anaerostipes hadrus, butyrate-producing bacterium SS2/1, is herein incorporated by reference in its entirety.

The content of DDBJ/EMBL/GENBANK accession number AJ315980.1 for microbial strain Anaerotruncus colihominis, culture collection DSM 17241, is herein incorporated by reference in its entirety.

The content of DDBJ/EMBL/GENBANK accession number AP009256.1 for microbial strain, Bifidobacterium adolescentis, culture collection ATCC 15703, is herein incorporated by reference in its entirety.

The content of DDBJ/EMBL/GENBANK accession number CP001095.1 for microbial strain Bifidobacterium longum subsp. infantis, culture collection ATCC 15697, is herein incorporated by reference in its entirety.

The content of DDBJ/EMBL/GenBank accession number U41172.1 for microbial strain Butyrivibrio fibrisolvens, culture collection ATCC 19171, is herein incorporated by reference in its entirety.

The content of DDBJ/EMBL/GenBank accession number AJ250365.2 for microbial strain Butyrivibrio fibrisolvens, 16.4, is herein incorporated by reference in its entirety.

The content of DDBJ/EMBL/GenBank accession number U41168.1 for microbial strain Butyrivibrio fibrisolvens, OB156, is herein incorporated by reference in its entirety.

The content of DDBJ/EMBL/GenBank accession number AY305305.1 for microbial strain Butyrate-producing bacterium, A2-232, is herein incorporated by reference in its entirety.

The content of DDBJ/EMBL/GenBank accession number AY305316.1 for microbial strain Butyrate-producing bacterium, SS3/4, is herein incorporated by reference in its entirety.

The content of DDBJ/EMBL/GENBANK accession number AE001437.1 for microbial strain Clostridium acetobutylicum, culture collection ATCC 824, is herein incorporated by reference in its entirety.

The content of DDBJ/EMBL/GENBANK accession number X78070.1 for microbial strain Clostridium acetobutylicum, culture collection DSM 792, is herein incorporated by reference in its entirety.

The content of DDBJ/EMBL/GENBANK accession number CP000721.1 for microbial strain Clostridium beijerinckii, culture collection NCIMB 8052, is herein incorporated by reference in its entirety.

The content of DDBJ/EMBL/GENBANK accession number X68189.1 for microbial strain Clostridium sporogenes, is herein incorporated by reference in its entirety.

The content of DDBJ/EMBL/GENBANK accession number X74770.1 for microbial strain Clostridium tetani, is herein incorporated by reference in its entirety.

The content of DDBJ/EMBL/GENBANK accession number AJ270491.2 for microbial strain Coprococcus, butyrate-producing bacterium L2-50, is herein incorporated by reference in its entirety.

The content of DDBJ/EMBL/GENBANK accession number EF031543.1 for microbial strain Coprococcus eutactus, culture collection ATCC 27759, is herein incorporated by reference in its entirety.

The content of DDBJ/EMBL/GenBank accession number AY305306.1 for microbial strain Eubacterium cylindroides, butyrate-producing bacterium T2-87, is herein incorporated by reference in its entirety.

The content of DDBJ/EMBL/GenBank accession number AY305313.1 for microbial strain Eubacterium cylindroides, butyrate-producing bacterium SM7/11, is herein incorporated by reference in its entirety.

The content of DDBJ/EMBL/GenBank accession number L34682.2 for microbial strain Eubacterium dolichum, culture collection DSM 3991, is herein incorporated by reference in its entirety.

The content of DDBJ/EMBL/GenBank accession number AJ270490.2 for microbial strain Eubacterium halii, butyrate-producing bacterium L2-7, is herein incorporated by reference in its entirety.

The content of DDBJ/EMBL/GenBank accession number AY305318.1 for microbial strain Eubacterium halii, butyrate-producing bacterium SM6/1, is herein incorporated by reference in its entirety.

The content of DDBJ/EMBL/GenBank accession number L34621.2 for microbial strain Eubacterium halii, culture collection ATCC 27751, is herein incorporated by reference in its entirety.

The content of DDBJ/EMBL/GenBank accession number AJ270475.2 for microbial strain Eubacterium rectale, A1-86, is herein incorporated by reference in its entirety.

The content of DDBJ/EMBL/GENBANK accession number NC_012781.1 for microbial strain Eubacterium rectale, culture collection ATCC 33656, is herein incorporated by reference in its entirety.

The content of DDBJ/EMBL/GenBank accession number L34421.2 for microbial strain Eubacterium ventriosum, culture collection ATCC 27560, is herein incorporated by reference in its entirety.

The content of DDBJ/EMBL/GENBANK accession number AY305307.1 for microbial strain Faecalibacterium prausnitzii, butyrate producing bacterium M21/2, is herein incorporated by reference in its entirety.

The content of DDBJ/EMBL/GENBANK accession number FP929046.1 for microbial strain Faecalibacterium prausnitzii is herein incorporated by reference in its entirety.

The content of DDBJ/EMBL/GENBANK accession number GG697168.2 for microbial strain Faecalibacterium prausnitzii is herein incorporated by reference in its entirety.

The content of DDBJ/EMBL/GENBANK accession number CP002158.1 for microbial strain Fibrobacter succinogenes subsp. succinogenes is herein incorporated by reference in its entirety.

The content of DDBJ/EMBL/GENBANK accession number NZ_AUJN01000001.1 for microbial strain Clostridium butyricum is herein incorporated by reference in its entirety.

The content of DDBJ/EMBL/GENBANK accession number NZ_AZUI01000001.1 for microbial strain Clostridium indolis, culture collection DSM 755, is herein incorporated by reference in its entirety.

The content of DDBJ/EMBL/GENBANK accession number ACEP01000175.1 for microbial strain Eubacterium hallii, culture collection DSM 3353, is herein incorporated by reference in its entirety.

The content of DDBJ/EMBL/GenBank accession number AY305310.1 for microbial strain Roseburia faecis, M72/1, is herein incorporated by reference in its entirety.

The content of DDBJ/EMBL/GenBank accession number AJ270482.2 for microbial strain Roseburia hominis, type strain A2-183T, is herein incorporated by reference in its entirety.

The content of DDBJ/EMBL/GenBank accession number AJ312385.1 for microbial strain Roseburia intestinalis, L1-82, is herein incorporated by reference in its entirety.

The content of DDBJ/EMBL/GenBank accession number AJ270473.3 for microbial strain Roseburia inulinivorans, type strain A2-194T, is herein incorporated by reference in its entirety.

The content of DDBJ/EMBL/GENBANK accession number NZ_ACFY01000179.1 for microbial strain Roseburia inulinivorans, culture collection DSM 16841, is herein incorporated by reference in its entirety.

The content of DDBJ/EMBL/GENBANK accession number K1912489.1 for microbial strain Ruminococcus flavefaciens, culture collection ATCC 19208, is herein incorporated by reference in its entirety.

The content of DDBJ/EMBL/GENBANK accession number AAYG02000043.1 for microbial strain Ruminococcus gnavus, culture collection ATCC 29149, is herein incorporated by reference in its entirety.

### BRIEF DESCRIPTION OF THE DRAWINGS

The patent application file contains at least one drawing executed in color. Copies of this patent or patent application with color drawing(s) will be provided by the Office upon request and payment of the necessary fee.

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIG. 1 depicts an illustrative flowchart for the methods disclosed herein.

FIG. 2 depicts illustrative microbiome-related health conditions and diseases for which metabolic maps and functional pathways can be determined, in accordance with disclosed embodiments. These health conditions can include: skin health, acne, atopic dermatitis, psoriasis, vaginosis, preterm delivery, allergies, preterm labor, chronic fatigue syndrome, Type 2 diabetes mellitus, depression, autism, asthma, hypertension, irritable bowel syndrome, metabolism, obesity, drug metabolism, Type I diabetes mellitus, multiple sclerosis, Clostridium difficile, inflammatory bowel disease, Crohn's disease, genitourinary disorders, or heart disease.

FIG. 3 depicts an illustrative butyrate pathway.

FIG. 4 exemplifies and compares performance of a method of pathway prediction of the present disclosure with other methods of pathway prediction.

FIG. 5 exemplifies the improvement in sensitivity and specificity values of an approach of the present disclosure (using a random forest model, denoted by a solid line, MCCV_RF) versus current methods of pathway prediction disclosed by Dale et al. (a machine learning classifier, denoted by a point with a circle icon; and a stated PathoLogic approach, denoted by a point with a triangle icon). The Monte Carlo cross-validation (MCCV) method was used for all three methods of pathway prediction.

FIG. 6 is an exemplary plot depicting Receiver Operating Characteristic (ROC) curves corresponding to (a) 8 different models, using Monte Carlo (MC) or leave-one-organism-out (L03) methods of cross-validation (CV), using logistic regression (logit) or random forest (rf), combined over the organisms in tier 1, and zoomed on the upper left portion of the plot; and (b) 2 different models described by Dale et al. (logistic regression and Pathologic prediction).

FIG. 7 is an exemplary plot depicting Receiver Operating Characteristic (ROC) curves corresponding to (a) 5 different models, using leave-one-organism-out (L03) cross-validation (CV), using each of logistic regression (logit) and random forest (rf), for each organism in tier 1; and (b) 2 different models described by Dale et al. (logistic regression and Pathologic prediction).

FIG. 8 is an exemplary plot depicting Receiver Operating Characteristic (ROC) curves corresponding to (a) models using leave-one-organism-out (LO3) cross-validation (CV), using each of logistic regression (logit) and random forest (rf), for each of the organisms in tiers 1, 2, and 3; and (b) 2 different models described by Dale et al. (logistic regression and Pathologic prediction). Two PGDBs have a smaller area under the curve (AUC) compared to those of Dale et al.: scocyc (worst) and mtbrvcyc (second worst).

FIG. 9 exemplifies protein functional annotation reference databases with controlled vocabularies.

FIG. 10 shows a computer system 1001 that is programmed or otherwise configured to implement methods provided herein.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the invention may be described in the context of a single embodiment, the features may also be provided separately or in any suitable combination. Conversely, although the invention may be described herein in the context of separate embodiments for clarity, the invention may also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

As used in the specification and claims, the singular forms “a,” “an,” and “the” include plural references unless the context clearly dictates otherwise. For example, the term “a sample” includes a plurality of samples, including mixtures thereof. In this application, the use of “or” means “and/or” unless stated otherwise. Furthermore, use of the term “including” as well as other forms, such as “include,” “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the inventions.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the invention can be used to achieve methods of the invention.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

The term “microbes,” “microorganisms” are used interchangeably herein and can refer to bacteria, archaea, eukaryotes (e.g. protozoa, fungi, yeast), and viruses, including bacterial viruses (i.e., phages).

The term “microbiome,” “microbiota,” and “microbial habitat” are used interchangeably herein and can refer to the ecological community of microorganisms that live on or in a subject's body. The microbiome can be comprised of commensal, symbiotic, and/or pathogenic microorganisms. Microbiomes can exist on or in many, if not most parts of the subject. Non-limiting examples of habitats of microbiome can include: body surfaces, body cavities, body fluids, the gut, the colon, skin, skin surfaces, skin pores, vaginal cavity, umbilical regions, conjunctival regions, intestinal regions, the stomach, the nasal cavities and passages, the gastrointestinal tract, the urogenital tracts, saliva, mucus, and feces. As used herein, the term “metagenome” or “metagenomic” refers to the collective genomes of all microorganisms present in the microbial habitat.

The term “prebiotic” as used herein can be a general term to refer to chemicals and/or ingredients that can affect the growth and/or activity of microorganisms in a host. Prebiotics can allow for specific changes in the composition and/or activity in the microbiome. Prebiotics can confer a health benefit on the host. Prebiotics can be selectively fermented, e.g., in the colon. Non-limiting examples of prebiotics can include: complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums (e.g., guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, P-glucan, and methylcellulose. Prebiotics can be found in foods, for example, acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, and banana. Prebiotics can be found in breast milk. Prebiotics can be administered in any suitable form, for example, capsule and dietary supplement.

The term “probiotic” as used herein can mean one or more microorganisms which, when administered appropriately, can confer a health benefit on the host or subject. Non-limiting examples of probiotics include, for example, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, Akkermansia, Bifidobacteria, Clostridia, Eubacteria, Verrucomicrobia, and Firmicutes.

The term “synbiotic” as used herein refers to a composition that contains both probiotics and prebiotics. A synbiotic composition beneficially affects a host by selectively stimulating the growth and/or activating the metabolism of one or more probiotic microorganisms in the host.

The terms “determining,” “measuring,” “evaluating,” “assessing,” “assaying,” and “analyzing” can be used interchangeably herein and can refer to any form of measurement, and include determining if an element is present or not (e.g., detection). These terms can include both quantitative and/or qualitative determinations. Assessing may be relative or absolute. These terms can include use of the algorithms and databases described herein. “Detecting the presence of” can include determining the amount of something present, as well as determining whether it is present or absent. The term “genome assembly algorithm” as used herein, refers to any method capable of aligning short reads with reference sequences under conditions that a complete sequence of the genome may be determined.

The term “genome” as used herein, can refer to the entirety of an organism's hereditary information that is encoded in its primary DNA sequence. The genome includes both the genes and the non-coding sequences. For example, the genome may represent a microbial genome or a mammalian genome. The genetic content of the microbiome can comprise: genomic DNA, RNA, and ribosomal RNA, the epigenome, plasmids, and/or all other types of genetic information found in the microbes that comprise the microbiome.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence.

The term “sequencing” as used herein refers to sequencing methods for determining the order of the nucleotide bases—adenine, guanine, cytosine, and thymine—in a nucleic acid molecule (e.g., a DNA or RNA nucleic acid molecule).

The term “biochip” or “array” can refer to a solid substrate having a generally planar surface to which an adsorbent is attached. A surface of the biochip can comprise a plurality of addressable locations, each of which location may have the adsorbent bound there. Biochips can be adapted to engage a probe interface, and therefore, function as probes. Protein biochips may be adapted for the capture of polypeptides and can be comprise surfaces having chromatographic or biospecific adsorbents attached thereto at addressable locations. Microarray chips are generally used for DNA and RNA gene expression detection.

The term “barcode” as used herein, refers to any unique, non-naturally occurring, nucleic acid sequence that may be used to identify the originating genome of a nucleic acid fragment.

The terms “subject,” “individual,” “host” or “patient” are used interchangeably herein and may refer to any animal subject, including humans, laboratory animals, livestock, and household pets. A subject can be a biological entity containing expressed genetic materials. The biological entity can be a microbe, including, e.g., bacteria, bacterial plasmids, viruses, fungi, and protozoa. A subject can host a variety of microorganisms. The subject can have different microbiomes in various habitats on and/or in his or her body. The subject may be diagnosed or suspected of being at elevated risk for a disease. The subject may have a microbiome state that is contributing to a disease (i.e., dysbiosis). In some cases, the subject is not necessarily diagnosed or suspected of being at elevated risk for the disease. In some instances a subject may be suffering from an infection or at risk of developing or transmitting to others an infection.

The terms “treatment” or “treating” are used interchangeably herein. These terms can refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect may include delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “16S,” “16S ribosomal subunit,” and “16S ribosomal RNA (rRNA)” can be used interchangeably herein and can refer to a component of a small subunit (e.g., 30S) of a prokaryotic (e.g., bacteria, archaea) ribosome. The 16S rRNA may be highly conserved evolutionarily among species of microorganisms. Consequently, sequencing of the 16S ribosomal subunit can be used to identify and/or compare microorganisms present in a sample (e.g., a microbiome).

The terms “23S,” “23 S ribosomal subunit,” and “23 S ribosomal RNA (rRNA)” can be used interchangeably herein and can refer to a component of a large subunit (e.g., 50S) of a prokaryotic (e.g., bacteria, archaea) ribosome. Sequencing of the 23S ribosomal subunit can be used to identify and/or compare microorganisms present in a sample (e.g., a microbiome).

The term “spore” can refer to a viable cell produced by a microorganism to resist unfavorable conditions such as high temperatures, humidity, and chemical agents. A spore can have thick walls that allow the microorganism to survive harsh conditions for extended periods of time. Under suitable environmental conditions, a spore can germinate to produce a living form of the microorganism that is capable of reproduction and all of the physiological activities of the microorganism.

### Overview

In some embodiments, the disclosure provides methods and compositions relating to determining metabolic maps and predicting functional pathways for customized microbial therapy.

### The Importance of the Human Microbiome

The human microbiome may be a factor in a variety of diseases, ranging from obesity and diabetes, to inflammatory bowel disease and cancer. A growing body of evidence indicates that the gut microbiome can play a central role in metabolic syndrome, which can bring serious health and cost burdens. For example, metabolic syndrome and related disorders have reached epidemic proportions within the United States. With an estimated 37% of the populace aged 20 years and above classified as prediabetic, the annual direct costs of diabetes alone in 2012 is estimated at $245 billion.

### Big Data Challenges of Microbiome Studies

A large amount of new microbiome-related data can overwhelm databases and toolsets. Metagenomic studies have resulted in an orders-of-magnitude increase in known protein families, and 16S rRNA marker gene surveys have increased the number of recognized microbial strains by orders of magnitude. While a host of new bioinformatic software and databases has been created, effective use of these systems may require significant investment both in bioinformatic training and local IT infrastructure. This represents a non-trivial loss of time and funds to microbiome field. These steep requirements of specialized bioinformatic training and computational facilities costs (for processing time and data storage) may present challenges to the field, both increasing the burden of and dissuading others from performing important analyses. Furthermore, many microbiome-related diseases may require complicated analyses of gene-expression data (transcriptomics or metabolomics) for discovering molecular mechanisms. Finally, there may be a lack of a standard database representation for the various microbiome-associated data formats, which can impede data integration, meta-analyses, and cross-study data mining.

### Microbiome Biochemistry

For many diseases mediated by the human microbiome, biochemistry plays a central role. Microbiome biochemistry may have a causal role in disease, such as that with artificial sweeteners and glucose intolerance and that with red meat consumption and cardiovascular disease. This underscores an importance of not only knowing which microbes are present in the human microbiome, but understanding the biochemical transformations which they facilitate. Therefore, it may be essential when studying such diseases to have a method to accurately predict metabolic pathways and their abundances from microbiome data (e.g. data generated from sequencing a population of microbes).

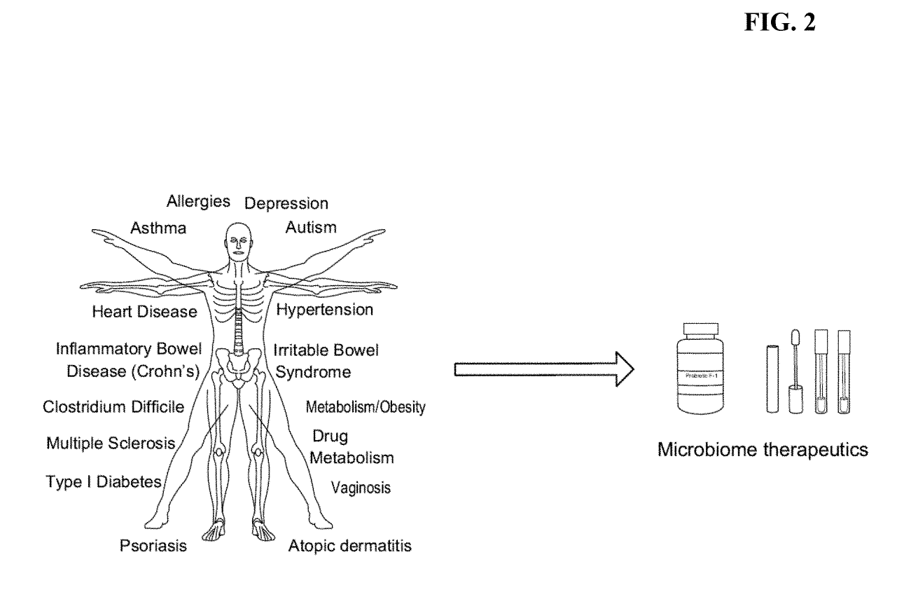

FIG. 2 depicts illustrative microbiome-related health conditions and diseases for which metabolic maps and functional pathways can be determined, in accordance with disclosed embodiments. These health conditions can include: skin health, acne, atopic dermatitis, psoriasis, vaginosis, preterm delivery, allergies, preterm labor, chronic fatigue syndrome, Type 2 diabetes mellitus, depression, autism, asthma, hypertension, irritable bowel syndrome, metabolism, obesity, drug metabolism, Type I diabetes mellitus, multiple sclerosis, Clostridium difficile, inflammatory bowel disease, Crohn's disease, genitourinary disorders, or heart disease. For such diseases, microbiome therapeutics may be identified and therapeutically effective doses of identified microbiome therapauetics may be administered to a subject diagnosed with, at risk of having, or suspecting of having, the microbiome-related health condition or disease.

### Role of Butyrate

Short-chain fatty acids (SCFAs), such as butyrate, can play a central role in modulating various body functions, as illustrated in FIG. 2. For example, butyrate can protect the brain and enhance plasticity in neurological diseases. Butyrate can serve an anti-inflammatory factor. Butyrate can affect gut permeability. Low levels of butyrate-producing microbes (e.g., Clostridium clusters XIVa and IV) and/or reduced lactate producing bacteria (e.g., Bifidobacterium adolescentis) can be correlated with, for example, gut dysbiosis, skin disorders, metabolic disorders, and behavioral/neurological disorders. Subsets of a formulation that comprise at least one primary fermenter and at least one secondary fermenter can be used for the treatment and/or mitigate progression of a disorder or condition.

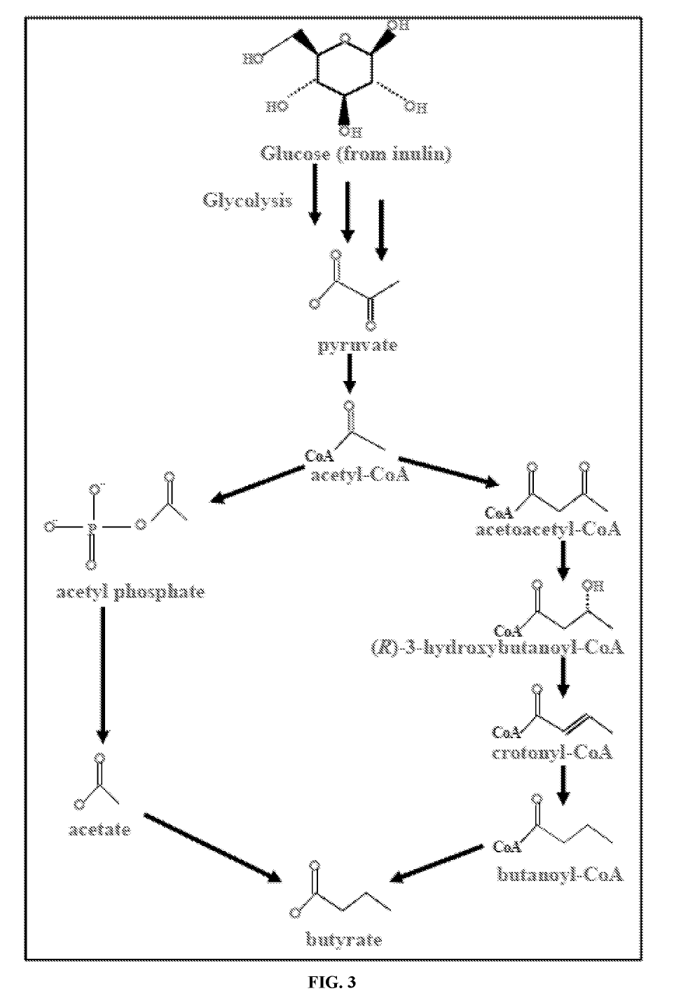

An illustrative butyrate pathway is illustrated in FIG. 3. In the colon, dietary fiber can be processed by butyrate-producing microorganisms to produce butyrate (i.e., butanoate), which is a short chain fatty acid (SCFA). In turn, butyrate can initiate G-protein coupled receptor (GPCR) signaling, leading to, for example, glucagon-like peptide-1 (GLP-1) secretion. GLP-1 can result in increased insulin sensitivity. Alteration of butyrate-producing microbiome in a subject can be associated with a disorder.

A composition may be administered to augment butyrate levels or production in a subject.

In some embodiments, the composition comprises a microbe with a butyrate kinase (e.g., EC 2.7.2.7; MetaCyc Reaction ID R11-RXN). Butyrate kinase is an enzyme that can belong to a family of transferases, for example those transferring phosphorus-containing groups (e.g., phosphotransferases) with a carboxy group as acceptor. The systematic name of this enzyme class can be ATP:butanoate 1-phosphotransferase. Butyrate kinase can participate in butyrate metabolism. Butyrate kinase can catalyze the following reaction:

ADP+butyryl-phosphate [symbol] ATP+butyrate

In some embodiments, the composition comprises a microbe with a Butyrate-Coenzyme A. Butyrate-Coenzyme A, also butyryl-coenzyme A, can be a coenzyme A-activated form of butyric acid. It can be acted upon by butyryl-CoA dehydrogenase and can be an intermediary compound in acetone-butanol-ethanol fermentation. Butyrate-Coenzyme A can be involved in butyrate metabolism.

In some embodiments, the composition comprises a microbe with a Butyrate-Coenzyme A transferase. Butyrate-Coenzyme A transferase, also known as butyrate-acetoacetate CoA-transferase, can belong to a family of transferases, for example, the CoA-transferases. The systematic name of this enzyme class can be butanoyl-CoA:acetoacetate CoA-transferase. Other names in common use can include butyryl coenzyme A-acetoacetate coenzyme A-transferase (e.g., EC 2.8.3.9; MetaCyc Reaction ID 2.8.3.9-RXN), and butyryl-CoA-acetoacetate CoA-transferase. Butyrate-Coenzyme A transferase can catalyze the following chemical reaction:

butanoyl-CoA+acetoacetate [symbol] butanoate+acetoacetyl-CoA

In some embodiments, the composition can comprise a microbe with an acetate Coenzyme A transferase (e.g., EC 2.8.3.1/2.8.3.8; MetaCyc Reaction ID BUTYRATE-KINASE-RXN).

In some embodiments, the composition comprises a microbe with a Butyryl-Coenzyme A dehydrogenase. Butyryl-CoA dehydrogenase can belong to the family of oxidoreductases, for example, those acting on the CH—CH group of donor with other acceptors. The systematic name of this enzyme class can be butanoyl-CoA:acceptor 2,3-oxidoreductase. Other names in common use can include butyryl dehydrogenase, unsaturated acyl-CoA reductase, ethylene reductase, enoyl-coenzyme A reductase, unsaturated acyl coenzyme A reductase, butyryl coenzyme A dehydrogenase, short-chain acyl CoA dehydrogenase, short-chain acyl-coenzyme A dehydrogenase, 3-hydroxyacyl CoA reductase, and butanoyl-CoA:(acceptor) 2,3-oxidoreductase. Non-limiting examples of metabolic pathways that butyryl-CoA dehydrogenase can participate in include: fatty acid metabolism; valine, leucine and isoleucine degradation; and butanoate metabolism. Butyryl-CoA dehydrogenase can employ one cofactor, FAD. Butyryl-CoA dehydrogenase can catalyze the following reaction:

butyryl-CoA+acceptor [symbol] 2-butenoyl-CoA+reduced acceptor

In some embodiments, the composition comprises a microbe with a beta-hydroxybutyryl-CoA dehydrogenase. Beta-hydroxybutyryl-CoA dehydrogenase or 3-hydroxybutyryl-CoA dehydrogenase can belong to a family of oxidoreductases, for example, those acting on the CH—OH group of donor with NAD+ or NADP+ as acceptor. The systematic name of the enzyme class can be (S)-3-hydroxybutanoyl-CoA:NADP+ oxidoreductase. Other names in common use can include beta-hydroxybutyryl coenzyme A dehydrogenase, L(+)-3-hydroxybutyryl-CoA dehydrogenase, BHBD, dehydrogenase, L-3-hydroxybutyryl coenzyme A (nicotinamide adenine, dinucleotide phosphate), L-(+)-3-hydroxybutyryl-CoA dehydrogenase, and 3-hydroxybutyryl-CoA dehydrogenase. Beta-hydroxybutyryl-CoA dehydrogenase enzyme can participate in benzoate degradation via co-ligation. Beta-hydroxybutyryl-CoA dehydrogenase enzyme can participate in butanoate metabolism. Beta-hydroxybutyryl-CoA dehydrogenase can catalyze the following reaction:

(S)-3-hydroxybutanoyl-CoA+NADP⁺ [symbol] 3-acetoacetyl-CoA+NADPH+H⁺

In some embodiments, the composition comprises a microbe with a crotonase. Crotonase can comprise enzymes with, for example, dehalogenase, hydratase, isomerase activities. Crotonase can be implicated in carbon-carbon bond formation, cleavage, and hydrolysis of thioesters. Enzymes in the crotonase superfamily can include, for example, enoyl-CoA hydratase which can catalyse the hydratation of 2-trans-enoyl-CoA into 3-hydroxyacyl-CoA; 3-2trans-enoyl-CoA isomerase or dodecenoyl-CoA isomerise (e.g., EC 5.3.3.8), which can shift the 3-double bond of the intermediates of unsaturated fatty acid oxidation to the 2-trans position; 3-hydroxbutyryl-CoA dehydratase (e.g., crotonase; EC 4.2.1.55), which can be involved in the butyrate/butanol-producing pathway; 4-Chlorobenzoyl-CoA dehalogenase (e.g., EC 3.8.1.6) which can catalyze the conversion of 4-chlorobenzoate-CoA to 4-hydroxybenzoate-CoA; dienoyl-CoA isomerase, which can catalyze the isomerisation of 3-trans,5-cis-dienoyl-CoA to 2-trans,4-trans-dienoyl-CoA; naphthoate synthase (e.g., MenB, or DHNA synthetase; EC 4.1.3.36), which can be involved in the biosynthesis of menaquinone (e.g., vitamin K2); carnitine racemase (e.g., gene caiD), which can catalyze the reversible conversion of crotonobetaine to L-carnitine in Escherichia coli; Methylmalonyl CoA decarboxylase (e.g., MMCD; EC 4.1.1.41); carboxymethylproline synthase (e.g., CarB), which can be involved in carbapenem biosynthesis; 6-oxo camphor hydrolase, which can catalyze the desymmetrization of bicyclic beta-diketones to optically active keto acids; the alpha subunit of fatty acid oxidation complex, a multi-enzyme complex that can catalyze the last three reactions in the fatty acid beta-oxidation cycle; and AUH protein, which can be a bifunctional RNA-binding homologue of enoyl-CoA hydratase.

In some embodiments, the composition comprises a microbe with a thiolase. Thiolases, also known as acetyl-coenzyme A acetyltransferases (ACAT), can convert two units of acetyl-CoA to acetoacetyl CoA, for example, in the mevalonate pathway. Thiolases can include, for example, degradative thiolases (e.g., EC 2.3.1.16) and biosynthetic thiolases (e.g., EC 2.3.1.9). 3-ketoacyl-CoA thiolase, also called thiolase I, can be involved in degradative pathways such as fatty acid beta-oxidation. Acetoacetyl-CoA thiolase, also called thiolase II, can be specific for the thiolysis of acetoacetyl-CoA and can be involved in biosynthetic pathways such as poly beta-hydroxybutyric acid synthesis or steroid biogenesis. A thiolase can catalyze the following reaction:

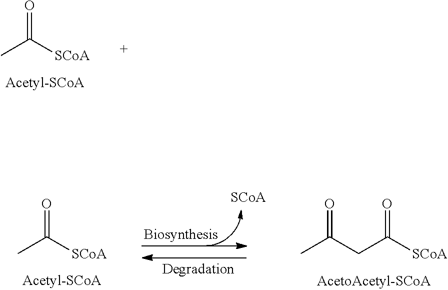

Production of butyrate can involve two major phases or microbes, for example, a primary fermenter and a secondary fermenter. The primary fermenter can produce intermediate molecules (e.g., lactate, acetate) when given an energy source (e.g., fiber). The secondary fermenter can convert the intermediate molecules produced by the primary fermenter into butyrate. Non-limiting examples of primary fermenter include Akkermansia muciniphila, Bifidobacterium adolescentis, Bifidobacterium infantis and Bifidobacterium longum. Non-limiting examples of secondary fermenter include Clostridium beijerinckii, Clostridium butyricum, Clostridium indolis, Eubacterium hallii, and Faecalibacterium prausnitzii. A combination of primary and secondary fermenters can be used to produce butyrate in a subject. Subsets of a formulation that comprises at least one primary fermenter and at least one secondary fermenter can be used for the treatment and/or mitigate progression of a metabolic health condition. The formulation can additionally comprise a prebiotic.

In some embodiments, a therapeutic composition comprises at least one primary fermenter and at least one secondary fermenter. In some embodiments, a therapeutic composition comprises at least one primary fermenter, at least one secondary fermenter, and at least one prebiotic. In one non-limiting example, a therapeutic composition can comprise Bifidobacterium adolescentis, Clostridium indolis, and inulin. In another non-limiting example, a therapeutic composition can comprise Bifidobacterium longum, Faecalibacterium prausnitzii, and starch.

Alterations in the relative abundance of SCFAs relative to each other can lead to a disorder. For example, an altered fiber-to-acetate production pathway or acetate-to-butyrate production pathway can lead to metabolic disorders such as bloating.

Akkermansia muciniphila can be a gram-negative, strict anaerobe that can play a role in mucin degradation. Akkermansia muciniphila can be associated with increased levels of endocannabinoids that control inflammation, the gut barrier, and gut peptide secretion. Akkermansia muciniphila can serve as a primary fermenter.

Bifidobacterium adolescentis can be a gram-positive anaerobe, which can be found in healthy human gut from infancy. Bifidobacterium adolescentis can synthesize B vitamins. Bifidobacterium adolescentis can serve as a primary fermenter.

Bifidobacterium infantis can be a gram-positive, catalase-negative, micro-aerotolerant anaerobe. Bifidobacterium infantis can serve as a primary fermenter.

Bifidobacterium longum can be a gram-positive, catalase-negative, micro-aerotolerant anaerobe. Bifidobacterium longum can serve as a primary fermenter.

Clostridium beijerinckii can be a gram-positive, strict anaerobe that belongs to Clostridial cluster I. Clostridium beijerinckii can serve as a secondary fermenter.

Clostridium butyricum can be a gram-positive, strict anaerobe that can serve as a secondary fermenter.

Clostridium indolis can be a gram-positive, strict anaerobe that belongs to Clostridial cluster XIVA. Clostridium indolis can serve as a secondary fermenter.

Eubacterium hallii can be a gram-positive anaerobe that belongs to Arrangement A Clostridial cluster XIVA. Eubacterium hallii can serve as a secondary fermenter.

Faecalibacterium prausnitzii can be a gram-positive anaerobe belonging to Clostridial cluster IV. Faecalibacterium prausnitzii can be one of the most common gut bacteria and the largest butyrate producer. Faecalibacterium prausnitzii can serve as a secondary fermenter.

Non-limiting examples of genes and/or proteins involved in the generation of butyrate include: butyryl-CoA dehydrogenase, beta-hydroxybutyryl-CoA dehydrogenase or 3-hydroxybutyryl-CoA dehydrogenase, crotonase, electron transfer protein a, electron transfer protein b, and thiolase. In some embodiments, the composition comprises a microbe with a gene or protein involved in SCFA (e.g., butyrate) production.

### Integrated Microbiome Analysis Platform

In some embodiments, the present disclosure provides methods to integrate metagenomic annotation system, metabolic pathway analysis, and biological dataset warehousing system into a microbiome platform. Integrated platforms can provide advanced metabolism representation, inference, search, visualization, analysis, and modeling capabilities to a microbiome analysis platform. The platform may further comprise a microbiome metabolic pathway prediction algorithm. A microbiome metabolic pathway prediction algorithm can comprise a machine learning algorithm. Algorithms can predict the distributed metabolic pathways present in the microbiome. An integrated platform can be available as a cloud-based platform. An integrated platform can be available as a web application.

In some embodiments, the present disclosure provides methods which can support functional annotation, metabolic reconstruction of metagenomic assemblies, and multi-omic data analysis on a cloud-based platform.

In some embodiments, the present disclosure provides method which can enable easy data storage and comparative analyses of thousands of microbiome samples in an open database.

In some embodiments, the present disclosure provides methods of predicting a microbiome metabolic pathway with technical advantages over other approaches (e.g., higher sensitivity, higher specificity, higher accuracy, higher AUC, or a combination thereof).

### Metagenomic Annotation Tools

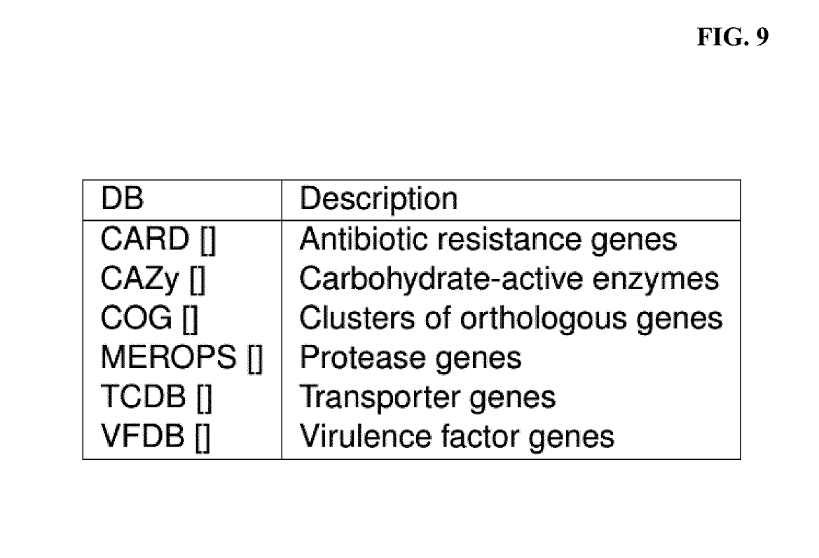

The integrated platform can have metagenomics annotation tools, such as MetaPathways, integrated therein. Microbiome studies can produce data describing relative abundance tables of OTUs, genes, or compounds. Data may be stored in biological observation matrix (BIOM) data format. The metagenomics annotication software can produce BIOM data files of gene abundances when provided with short read data from a metagenomic or metatranscriptomic shotgun sequencing corresponding to the assembly input. The short reads can be aligned to the assembly to compute the gene coverage, and thus the gene abundance. Gene abundances can be reported relative to matching genes in a protein reference database such as SwissProt, so that different samples can be compared to one another. Use of the BIOM data standard can increase the interoperability of the integrated platform disclosed herein with other software, and can support the wider microbiome bioinformatics ecosystem. A platform can have additional software integrated to normalize the raw gene counts prior to BIOM file generation. The software can be MicrobeCensus. Metagnomic sequences reads can be annotated and stored in reference databases with controlled vocabularies. Examples of exemplary controlled vocabularies are provided in FIG. 9.

### Metabolic Pathway Analysis Tools

Metabolic pathway analysis tools may be used to construct metabolic pathways in view of annotated sequence reads (e.g., taxonomic annotations). These tools may be incorporated into an integrated platform. An integrated platform can have Pathway Tools integrated. An integrated platform can have MetaPathways and Pathway Tools integrated. After MetaPathways has successfully annotated a genome or metagenome, it can produce input files for Pathway Tools to build an environmental Pathway/Genome Database (ePGDB), or “metabolic reconstruction.” In some instances, the ePGDB schema can be extended to support an attribute associated with a gene object, called TAXONOMIC-ANNOT. This feature can store the gene taxonomic annotation generated using MetaPathways, for example, via the LCA method of determining the most likely phylogenetic placement of a gene (e.g., taxonomic assignment within a genome) given several sequence similarity matches to a reference database. Given input annotation data with the TAXONOMIC-ANNOT attribute set, the PathoLogic module of Pathway Tools may incorporate the attribute into the ePGDB gene objects. This may enable powerful taxonomic queries and visualizations in Pathway Tools. For example, implementation of a Pathway Tools web API call that specifies a high-level taxon ID from the NCBI Taxonomy Database, such as phylum Firmicutes (Taxon ID: 1239), and all corresponding reactions on the Cellular Overview (a metabolic map visualization) with enzymes encoded by genes with a taxonomic annotation of Firmicutes (or a sub-taxon of Firmicutes), can be highlighted, thereby allowing for determination of which portions of the predicted metabolic network correspond to which taxa.

### Microbiome Samples

In some embodiments, the methods disclosed herein can be used to analyze any sample that has a microbiome. The sample may be a biological sample. The sample may be an environmental sample.

### Biological Samples

A biological sample can be collected from a subject to determine the microbiome profile of the subject. Non-limiting examples of subject include humans, laboratory animals, livestock, and household pets. A subject can be a biological entity containing expressed genetic materials. The biological sample can be any sample type from any microbial habitat on the body of a subject. Non-limiting examples of microbial habitats include skin habitat, umbilical habitat, vaginal habitat, amniotic fluid habitat, conjunctival habitat, intestinal habitat, stomach habitat, gut habitat, oral habitat, nasal habitat, gastrointestinal tract habitat, respiratory habitat, and urogenital tract habitat.

Depending on the application, the selection of a biological sample can be tailored to the specific application. The biological sample can be for example, whole blood, serum, plasma, mucosa, saliva, cheek swab, urine, stool, cells, tissue, bodily fluid, lymph fluid, CNS fluid, and lesion exudates. A combination of biological samples can be used with the methods of the disclosure.

### Environmental Samples

An environmental sample can be collected to determine the microbiome profile. The environmental sample can be any sample type from any microbial habitat in the environment. The environmental sample can be an agricultural sample. The environmental sample can be an oceanic sample. Non-limiting examples of environmental microbial habitats include, but are not limited to, soil sample, water samples, plant tissue, sewage samples, urban environment sampling, built environment sampling, dirt, and debris filtered from the air.

Depending on the application, the selection of an environmental sample can be tailored to the specific application. A combination of environmental samples can be used with the methods of the disclosure.

### Sample Preparation

Sample preparation can comprise any one of the following steps or a combination of steps. A sterile swab may be first dipped into a tube containing sterile phosphate buffered saline (PBS) to wet. The swab may be swiped across the area of interest multiple times (e.g., 10-20 times). The swab may be gently dipped into a buffer (e.g., a lysis buffer) in a sterile tube. The swab may be left in the tube for shipping to a laboratory to be further analyzed as provided herein. The samples obtained can be shipped overnight at room temperature. Shipping microbial cells in buffers can introduce detection bias in the samples. Some microbes can continue propagating on the nutrients that come along with sample collection. Some microbes can undergo apoptosis in the absence of a specific environment. As a result, microbial samples shipped in this fashion can have an initial profiling/population bias associated with cellular integrity.

Methods can be used to enrich intact cells by first centrifuging the collected sample. The resulting pellet, formed from the intact cells within the sample, can then be used as a precursor for all of the downstream steps. In some embodiments, the methods of the present disclosure further comprise a purification step to concentrate any DNA present in the supernatant (e.g., from already lysed cells). This DNA can be combined with DNA extracted from a standard pellet preparation. The combined DNA can form a more complete precursor to downstream steps.

Cell lysis and/or extraction of nucleic acids from the cells can be performed by any suitable methods, including physical methods, chemical methods, or a combination of both. Nucleic acids can be isolated from a biological sample using shearing methods, which preserve the integrity and continuity of genomic DNA.

A nucleic acid sample used with methods of the present disclosure can include any type of DNA and/or RNA. The length of nucleic acids can be about 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, 10,000,000, or more than 10,000,000 nucleotides or base pairs in length.

An amplicon approach can be used to prepare DNA for microbiome profiling. This approach can comprise a number of steps, for example, PCR, sample quantification (e.g., Qubit, nanodrop, bioanalyzer, etc.), Blue Pippin size selection, 0.5× Ampure purification, sample quantification, DNA end repair, 0.5× Ampure purification, blunt end adaptor ligation, exo-nuclease treatment, two 0.5× Ampure purifications, and final Blue Pippen size selection.

In some embodiments, the method does not comprise amplification. Examples of such methods include preparation of samples for sequencing by Whole Genome Shotgun (WGS) sequencing. These approaches can provide a benefit by removing amplification bias that can skew microbial distributions. In addition, such approaches can allow for de novo discovery of pertinent elements, for example, bacterial plasmids, fungi, and viruses.

Methods of the present disclosure can employ conventional techniques of immunology, biochemistry, chemistry, molecular biology, microbiology, cell biology, genomics, and/or recombinant DNA, which are within the skill of the art. For example, preparation of a sample can comprise, e.g., extraction or isolation of intracellular material from a cell or tissue such as the extraction of nucleic acids, protein, or other macromolecules. Sample preparation approaches which can be used with methods of the present disclosure include but are not limited to, centrifugation, affinity chromatography, magnetic separation, immunoassay, nucleic acid assay, receptor-based assay, cytometric assay, colorimetric assay, enzymatic assay, electrophoretic assay, electrochemical assay, spectroscopic assay, chromatographic assay, microscopic assay, topographic assay, calorimetric assay, radioisotope assay, protein synthesis assay, histological assay, culture assay, and combinations thereof.

The sample preparation, DNA extraction, and sequencing of the sample may be performed using methods appropriate to the samples and known in the art.

### Data Input

### Metagenomics

The integrated platform can accept as input assembled metagenomic sequences. The integrated platform can accept as input unassembled metagenomic sequences. The integrated platform can accept as input transcriptomic data. The integrated platform can accept as input a metagenomic assembly and raw metagenomic shotgun reads. The integrated platform can accept as input a 16S rRNA dataset. Exemplary methods used to obtain the sequences for the platform include but are not limited to Shotgun sequencing, Sanger sequencing, pyrosequencing, 16S rRNA sequencing, and RNA-seq.

### Metabolomics

The integrated platform can support metabolomics data. Exemplary methods used to obtain metabolomics data for the platform include but are not limited to gas-chromatography-mass spectrometry (GC-MS), NMR spectroscopy, and mass spectrometry.

### Proteomics

The integrated platform can support proteomics data. Exemplary methods used to obtain proteomics data for the platform include but are not limited to mass spectrometry, matrix-assisted laser desorption/ionization (MALDI), electrospray ionization (ESI), protein microarrays, and reverse phase protein microarrays.

### Microbiome Metabolic Pathway Prediction

In some instances, the present disclosure provides a method of predicting metabolic pathways (e.g., microbiome metabolic pathways). The microbiome metabolic pathway prediction algorithm can comprise machine learning. In some embodiments, the present disclosure provides methods of predicting metabolic pathways in microbiome samples, with improved accuracy compared to other methods, and/or determining or estimating the abundance (e.g., relative or normalized) of pathways. Sequences encoding a component of a metabolic pathway in a genome may be obtained from sequencing information of nucleic acid molecules from a population of organisms (e.g. in a biological sample). From the sequencing information, a presence of a nucleic acid marker that encodes a component of the metabolic pathway in a genome of each of one or more organisms (e.g., microbes) in a plurality of different organisms (e.g., microbes) in the population may be detected. Presence of a nucleic acid marker (e.g., which encodes the component of the metabolic pathway in the genome of the organism) may be determined by identifying an organism in the population based on the sequencing information, identifying a set of reactions for the organism, and determining the presence of the nucleic acid marker from the organism in the identified set of reactions. An abundance of a metabolic pathway may be based in part on the presence or amount of sequences encoding a component of a metabolic pathway in a genome.

The method of predicting microbiome metabolic pathway may be a technical improvement over other approaches to performing microbiome pathway prediction due at least in part to 1) improved accuracy, 2) being able to provide normalized pathway abundances, and 3) providing the relative significance of the pathways present. Exemplary results provided from a pathway prediction algorithm disclosed herein (DogPath) are provided in Table 1.

The microbiome metabolic pathway prediction algorithm may analyze acquired genomic information (e.g., sequencing data, contigs, and/or genomic assemblies) from known microbes to generate an output of a likelihood of presence/absence or abundance of the pathway. The prediction algorithm may comprise an artificial intelligence based predictor, such as a machine learning based predictor, configured to process the acquired genomic information to generate the output of a likelihood of presence/absence or abundance of the pathway. The machine learning predictor may be trained using datasets comprising genomic information from one or more sets of known microbes as inputs and known or likely measurements or determinations of presence/absence or abundance of the pathway as outputs to the machine learning predictor.

The machine learning predictor may comprise one or more machine learning algorithms. Examples of machine learning algorithms may include a support vector machine (SVM), a naïve Bayes classification, a logistic regression, a random forest, a neural network, deep learning, or other supervised learning algorithm or unsupervised learning algorithm for classification and regression. The machine learning predictor may be trained using one or more training datasets corresponding to genomic information of known microbes.

Training datasets may be generated from, for example, one or more sets of known microbes having known features and known presence/absence or abundance (labels). Training datasets may comprise a set of features and labels corresponding to the features. Features may comprise characteristics of the genomic information, as described elsewhere herein. For example, a set of features collected from a known microbe may collectively serve as a presence/absence or abundance signature, which may be indicative of presence or absence of a nucleic acid marker (e.g., which encodes a component of the metabolic pathway in a genome of the microbe). Such nucleic acid markers may be indicative of a presence/absence or abundance of the microbe in a sample.

Training sets (e.g., training datasets) may be selected by random sampling of a set of data corresponding to one or more sets of known microbes. Alternatively, training sets (e.g., training datasets) may be selected by proportionate sampling of a set of data corresponding to one or more sets of known microbes. The machine learning predictor may be trained until certain predetermined conditions for accuracy or performance are satisfied, such as having minimum desired values corresponding to prediction metrics. For example, the prediction metric may correspond to prediction of a presence/absence or abundance of a nucleic acid marker or a microbe in a sample. Examples of prediction metrics may include sensitivity, specificity, positive predictive value (PPV), negative predictive value (NPV), accuracy, area under the curve (AUC) of a Receiver Operating Characteristic (ROC) curve, or any expected value (e.g., mean or median) thereof, corresponding to the presence/absence or abundance of the nucleic acid marker or the microbe in a sample.

As another example, such a predetermined condition may be that the sensitivity of identifying the presence/absence or predicting the abundance of a nucleic acid marker or microbe comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the specificity of identifying the presence/absence or predicting the abundance of a nucleic acid marker or microbe comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the positive predictive value (PPV) of identifying the presence/absence or predicting the abundance of a nucleic acid marker or microbe comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the negative predictive value (NPV) of identifying the presence/absence or predicting the abundance of a nucleic acid marker or microbe comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the area under the curve (AUC) of a Receiver Operating Characteristic (ROC) curve of identifying the presence/absence or predicting the abundance of a nucleic acid marker or microbe comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

### Improved Accuracy

In some embodiments the present disclosure provides methods of predicting metabolic pathways in microbiome samples, which methods are a technical improvement upon the accuracy of other methods. The method of predicting microbiome metabolic pathway may have an improved accuracy of at least about 99.8%. The improved accuracy can be at least about 99%. The improved accuracy can be at least about 98%. The improved accuracy can be at least about 97%. The improved accuracy can be at least about 96%. The improved accuracy can be at least about 95%. The improved accuracy can be at least about 94%. The improved accuracy can be at least about 93%. The improved accuracy can be at least about 92%. The improved accuracy can be at least about 91%. The method of predicting microbiome metabolic pathway disclosed herein may have an improved accuracy between about 99.8% to about 98%. The improved accuracy can be between about 98% to about 96%. The improved accuracy can be between about 96% to about 94%. The improved accuracy can be between about 94% to about 92%. The improved accuracy can be between about 92% to about 91%. The improved accuracy can be between about 91% to about 90%.

### Pathway Abundances and its Relative Significance

For determining the metabolic role of the human microbiome in diseases such as obesity, it may be critical to determine which metabolic pathways are present. Tools such as IMG/M and MG-RAST may annotate metagenomes with enzyme annotations, but may not provide any prediction of which pathways associated with the enzymes are present. Results of such annotation can artificially inflate predictions regarding a number of pathways present in a sample. Pathway abundance problems can be decomposed into two parts: 1) computing strain abundance, and 2) predicting the metabolic pathways of each strain. Parsimony methods like MinPath may predict the minimal number of pathways necessary to explain the observed enzymes, but may fail to predict pathways consisting of enzymes also found in other pathways. Finally, other methods may not take enzyme abundance into account, which may render them susceptible to false positives due to trace amounts of enzymes being detected. Also, predicting pathway abundances (e.g., abundances of nucleic acid markers) may permit more meaningful analysis of samples and inter-sample comparison than merely predicting sets of pathways (e.g., nucleic acid markers) which are present.

Microbiome pathway prediction can predict microbiome pathways associated with the enzymes present in a given microbiome sample. Microbiome pathway prediction can predict pathway abundances abundances (e.g., abundances of nucleic acid markers) based on the enzyme abundance present in a given microbiome sample. The observed enzyme abundances can be formally modeled as a linear mixture model of the organisms present. The metabolic pathway may be determined using a model trained using metabolic pathway data that is tiered, with each tier corresponding to a different confidence level in the metabolic data. The methods can further comprise generating one or more feature vectors being indicative of a metabolic pathway.

### Machine Learning Methods for Functional Annotation and Microbiome Metabolic Pathway Prediction

Machine learning methods may be used to predict a metabolic pathway associated with a microbe in a population of microbes. For example, neural networks such as deep learning neural networks may be incorporated into a machine learning approach to tackle the problem of predicting “distributed pathways” among a complex mixture of bacteria along with more accurate protein function prediction.

### Functional Annotation of Proteins

Errors in pathway prediction may not stem from the pathway prediction algorithm itself, but “upstream” in the functional predictions of the genome's proteins. Predicting pathways accurately may be challenging if there are inaccurate predictions of the enzymes that catalyze the pathway's reactions. In analyzing the best-in-class methods as determined by the Critical Assessment of Functional Annotation (CAFA) competitions, Blast-based assignment (e.g., as used by MetaPathways) may be sub-optimal compared to better performing methods. A machine learning method (e.g., deep learning) can be applied to the problem of functional annotation, focusing on the accurate prediction of enzymes.

### Microbiome Distributed Pathway Prediction

Distributed pathways can be metabolic paths through a consortium of organisms in a microbiome; achieving an overall metabolic transformation that no one organism may be capable of. For example, in a true distributed pathway, there may be microbial transporters for intermediate metabolites to allow part of a pathway to begin in one organism, and then end in another through export and then import of the metabolite. The microbiome pathway prediction methods may be extended to incorporate machine learning (e.g., deep learning) to be able to accurately predict distributed pathways.

In some embodiments, the integration of machine learning (e.g., deep learning) into the methods to predict microbiome metabolic pathway represents a technical advancement over other methods due at least in part to an ability to correct functional annotation of proteins upstream, thereby allowing more accurate prediction of pathways present. The improved functional annotation may accurately predict enzymes. The improved functional annotation may accurately predict transport proteins. The integration of machine learning (e.g., deep learning) into the methods to predict microbiome metabolic pathway may represent a technical advancement over other methods due at least in part to an ability to not only predict metabolic pathways but also microbiome distributed pathways to determine the overall metabolic transformation in a subject.

### Time-Series Analysis for Predicting Metabolic Pathway Changes

In some embodiments, the methods disclosed herein further have a time-series algorithm integrated into the platform. The time-series analysis can predict metabolic pathways changes of one or more microbes in the microbiome over time. The samples collected for time-series analysis can be at multiple time points from the same source. The samples collected for time-series analysis can be at multiple time points from multiple sources. For example, samples can be collected every day for a month. Samples can be collected every 2 days for a month. Samples can be collected every 3 days for a month. Samples can be collected every 4 days for a month. Samples can be collected every 5 days for a month. Samples can be collected every 6 days for a month. Samples can be collected every 7 days for a month. Samples can be collected every day over 2 months. Samples can be collected every 2 days over 2 months. Samples can be collected every 3 days over 2 months. Sample can be collected every 4 days over 2 months. Samples can be collected every 5 days over 2 months. Samples can be collected every 6 days over 2 months. Samples can be collected every 7 days over 2 months. Samples can be collected every day over 3 months. Samples can be collected every 2 days over 3 months. Samples can be collected every 3 days over 3 months. Samples can be collected every 4 days over 3 months. Samples can be collected every 5 days over 3 months. Samples can be collected every 6 days over 3 months. Samples can be collected every 7 days over 3 months. A sample collection regimen can continue over a one-month period. A sample collection regimen can continue over a two-month period. A sample collection regimen can continue over a three-month period. A sample collection regimen can continue over a four-month period. A sample collection regimen can continue over a five-month period. A sample collection regimen can continue over a six-month period. A sample collection regimen can be followed every alternate month. A sample collection regimen can be followed every 2 months. A sample collection regimen can be followed every 3 months. A sample collection regimen can be followed every 4 months. A sample collection regimen can be followed every 5 months. A sample collection regimen can be followed every 6 months. Samples can be collected before or after a composition comprising population of microbes has been administered. Samples can be collected before and after a composition comprising population of microbes has been administered. Samples can be collected over time to determine microbiome metabolic pathway changes that are affected due to changes in environment. In some instances, a time-series profile of the microbiome can be generated. The time-series analysis used can comprise a time-decay. The time-series analysis used can be detrending. The time-series analysis used can comprise an augmented-Dickey Fuller test. The time-series analysis used can comprise a cross-correlation. The time-series analysis used can comprise a local similarity analysis (LSA). The time-series analysis used can comprise a time-varying network inference. The time-series analysis used can comprise an auto-correlation. The time-series analysis used can comprise an auto-correlogram. The time-series analysis used can comprise a Hurst exponent. The time-series analysis used can comprise a Lyapunov exponent. The time-series analysis used can comprise a predictability analysis. The time-series analysis used can comprise a bistability analysis. The time-series analysis used can comprise early warning signs. The time-series analysis used can comprise a combination of one or more of the methods.

### Methods for Determining Microbiome Metabolic Maps

The present disclosure provides methods and compositions comprising microbial populations for the treatment of microbiome-related health conditions and/or disorders in a subject based on a microbiome metabolic map. In some embodiments, the methods disclosed herein comprise using microbiome profiling and metabolite profiling to determine a microbiome metabolic map. In some embodiments, the method comprises detecting a population of microbes in a subject responsive to administration of a composition comprising one or more butyrate-producing microbes, the method comprising: (a) obtaining a biological sample from the subject comprising a population of microbes and one or more metabolites; (b) assaying the biological sample to identify one or more metabolites; and (c) detecting one or more metabolic maps indicative of responsiveness to the composition comprising one or more butyrate-producing microbes. In some embodiments, the metabolic map can include normal butyrate levels in sample and normal levels of strains which can produce carbohydrates and sugars that feed butyrate producing strains. In some embodiments, the metabolic map can include normal butyrate levels in sample and reduced levels of strains which can produce carbohydrates and sugars that feed butyrate producing strains. In some embodiments, the metabolic map can include reduced butyrate levels in sample and normal levels of strains which can produce carbohydrates and sugars that feed butyrate producing strains. In some embodiments, the metabolic map can include reduced butyrate levels in sample and reduced levels of strains which can produce carbohydrates and sugars that feed butyrate producing strains. Methods of the disclosure can include collection, stabilization and extraction of microbes for microbiome analysis. Methods of the disclosure can include determining the microbiome profile of any suitable microbial habitat of the subject. The composition of the microbial habitat can be used to diagnose a health condition of a subject, for example, to determine likelihood of a disorder and/or treatment course of the disorder.

An exemplary method of the disclosure can comprise at least one of the following steps: obtaining a sample from a subject, measuring a panel of microbes in the sample and the metabolite profile associated to determine a metabolic map, comparing the metabolic map in the sample with metabolic maps of the microbes found in a healthy sample, determining status of a disease upon the measuring, generating a report that provides information of disease status upon the results of the determining, and administering microbial-based compositions of the disclosure to the subject for treating a disorder such as a microbiome-based disorder, or the presence or absence of a microbe or metabolite.

Methods for profiling a microbiome are discussed in U.S. patent application Ser. No. 14/437,133, which is incorporated herein by reference in its entirety for all purposes.

Detection methods, for example, long read sequencing, can be used to profile a microbiome and/or identify microbiome biomarkers.

Microbiomes from, for example, body cavities, body fluids, gut, colon, vaginal cavity, umbilical regions, conjunctival regions, intestinal regions, the stomach, the nasal cavities and passages, the gastrointestinal tract, the urogenital tracts, saliva, mucus, and feces, can be analyzed and compared with that of control (e.g., healthy or diseased) subjects. An increased and/or decreased diversity of gut microbiome can be associated with a disorder. Subjects with a disorder can have a lower prevalence of butyrate-producing bacteria, for example, C. eutactus.

In some embodiments, methods of the present disclosure can be used to determine microbial habitat of the gut or gastrointestinal tract of a subject. The gut comprises a complex microbiome including multiple species of microbes that can contribute to vitamin production and absorption, metabolism of proteins and bile acids, fermentation of dietary carbohydrates, and prevention of pathogen overgrowth. The composition of microbes within the gut can be linked to functional metabolic pathways in a subject. Non-limiting examples of metabolic pathways linked to gut microbiota include energy balance regulation, secretion of leptin, lipid synthesis, hepatic insulin sensitivity, modulation of intestinal environment, and appetite signaling. Modification (e.g., dysbiosis) of the gut microbiome can increase the risk for health conditions such as diabetes, mental disorders, ulcerative colitis, colorectal cancer, autoimmune disorders, obesity, diabetes, and inflammatory bowel disease.

In some embodiments, detection methods (e.g., sequencing) can be used to identify microbiome biomarkers associated with a disease or disorder.

In some embodiments, detection methods of the disclosure (e.g., sequencing) can be used to analyze changes in microbiome composition over time, for example, during antibiotic treatment, microbiome therapies, and various diets. The microbiome can be significantly altered upon exposure to antibiotics and diets that deplete the native microbial population. Methods of the present disclosure can be used to generate profiles of the subject before and after administration of a therapeutic to characterize differences in the microbiota.

In some embodiments, methods to visualize the microbiome based on sequencing signatures are provided. In some embodiments, methods are provided to visualize the microbiome over time based on sequencing information.

Methods of the disclosure can be used to detect, characterize, and quantify microbial habitat of a subject. The microbial habit can be used to define the diversity and abundance of microbes in order to evaluate clinical significance and causal framework for a disorder. Microbiome profiles can be compared to determine microbes that can be used as biomarkers for predicting and/or treating a health condition.

### Microbiome Profiling

The present disclosure provides methods for measuring at least one microbe in a biological sample from at least one microbial habitat of a subject and determining a microbiome profile. A microbiome profile can be assessed using any suitable detection means that can measure or quantify one or more microbes (e.g., bacteria, fungi, viruses, and archaea) that comprise a microbiome. The microbiome profile may be determined using metagenomic sequencing. Exemplary methods used to obtain the sequences for the microbiome profile include but are not limited to shotgun sequencing, Sanger sequencing, pyrosequencing, and 16S rRNA sequencing.

Nucleic acid sample prepared from a biological sample can be subjected to a detection method to generate a profile of the microbiome associated with the sample. Profiling of a microbiome can comprise one or more detection methods.

Methods of the present disclosure can be used to measure, for example, a 16S ribosomal subunit, a 23 S ribosomal subunit, intergenic regions, and other genetic elements. Suitable detection methods can be chosen to provide sufficient discriminative power in a particular microbe in order to identify informative microbiome profiles.

In some embodiments, a ribosomal RNA (rRNA) operon of a microbe is analyzed to determine a subject's microbiome profile. In some embodiments, the entire genomic region of the 16S or 23S ribosomal subunit of the microbe is analyzed to determine a subject's microbiome profile. In some embodiments, the variable regions of the 16S and/or 23S ribosomal subunit of the microbe are analyzed to determine a subject's microbiome profile.

In some embodiments, the entire genome of the microbe is analyzed to determine a subject's microbiome profile. In other embodiments, the variable regions of the microbe's genome are analyzed to determine a subject's microbiome profile. For example, genetic variation in the genome can include restriction fragment length polymorphisms, single nucleotide polymorphisms, insertions, deletions, indels (insertions or deletions), microsatellite repeats, minisatellite repeats, short tandem repeats, transposable elements, randomly amplified polymorphic DNA, amplification fragment length polymorphism, or a combination thereof.

In some embodiments, sequencing methods such as long-read length single molecule sequencing is used for detection. Long read sequencing can provide microbial classification down to the strain resolution of each microbe. Examples of sequencing technologies that can be used with the present disclosure for achieving long read lengths include the SMRT sequencing systems from Pacific Biosciences, long read length Sanger sequencing, long read ensemble sequencing approaches, e.g., Illumina/Moleculo sequencing and potentially, other single molecule sequencing approaches, such as Nanopore sequencing technologies.

Long read sequencing can include sequencing that provides a contiguous sequence read of, for example, longer than 500 bases, longer than 800 bases, longer than 1000 bases, longer than 1500 bases, longer than 2000 bases, longer than 3000 bases, or longer than 4500 bases.

In some embodiments, detection methods of the present disclosure comprise amplification-mode sequencing to profile the microbiome. In some embodiments, detection methods of the disclosure comprise a non-amplification mode, for example, Whole Genome Shotgun (WGS) sequencing, to profile the microbiome.

Primers used in methods of the present disclosure can be prepared by any suitable method, for example, cloning of appropriate sequences and direct chemical synthesis. Primers can also be obtained from commercial sources. In addition, computer programs can be used to design primers. Primers can contain unique barcode identifiers.

Microbiome profiling can further comprise use of, for example, a nucleic acid microarray, a biochip, a protein microarray, an analytical protein microarray, reverse phase protein microarray (RPA), a digital PCR device, and/or a droplet digital PCR (ddPCR) device.

In some embodiments, the microbial profile is determined using additional information such as age, weight, gender, medical history, risk factors, family history, or any other clinically relevant information. In some embodiments, a subject's microbiome profile can comprise at least 1, 2, 3, 4, 5, 6, 7, 8, 9, 10, 11, 12, 13, 14, 15, 16, 17, 18, 19, 20, or more than 20 microbiomes.

A subject's microbiome profile can comprise one microbe. In some embodiments, a subject's microbiome profile comprises, for example, 2 microbes, 3 or fewer microbes, 4 or fewer microbes, 5 or fewer microbes, 6 or fewer microbes, 7 or fewer microbes, 8 or fewer microbes, 9 or fewer microbes, 10 or fewer microbes, 11 or fewer microbes, no more than 12 microbes, 13 or fewer microbes, 14 or fewer microbes, 15 or fewer microbes, 16 or fewer microbes, 18 or fewer microbes, 19 or fewer microbes, 20 or fewer microbes, 25 or fewer microbes, 30 or fewer microbes, 35 or fewer microbes, 40 or fewer microbes, 45 or fewer microbes, 50 or fewer microbes, 55 or fewer microbes, 60 or fewer microbes, 65 or fewer microbes, 70 or fewer microbes, 75 or fewer microbes, 80 or fewer microbes, 85 or fewer microbes, 90 or fewer microbes, 100 or fewer microbes, 200 or fewer microbes, 300 or fewer microbes, 400 or fewer microbe, 500 or fewer microbes, 600 or fewer microbes, 700 or fewer microbes, or 800 or fewer microbes.

### Metabolite Profiling

In some instances, the present disclosure provides methods of determining a metabolite profile of a microbiome. In some cases, metabolite profiling can comprise analysis of a group of metabolites that is related to a specific metabolic pathway. The metabolic pathway may be a butyrate pathway. Methods used to determine metabolite profile can include, for example, liquid chromatography-mass spectrometry (LC-MS), Gas chromatography-mass spectrometry (GC-MS), Liquid Chromatography with Nuclear Magnetic Resonance Spectroscopy (LC-NMR), and Nuclear magnetic resonance spectroscopy (NMR).

### Liquid Chromatography-Mass Spectrometry (LC-MS)

Liquid chromatography-mass spectrometry (LC-MS) can combine physical separation capabilities of liquid chromatography (or HPLC) with mass analysis capabilities of mass spectrometry (MS). While liquid chromatography separates mixtures with multiple components, mass spectrometry may provide structural identity of the individual components with high molecular specificity and detection sensitivity. This tandem technique can be used to analyze biochemical, organic, and inorganic compounds commonly found in complex samples of environmental and biological origin. In some instances, metabolite profiling of the microbiome is determined using liquid chromatography-mass spectrometry (LC-MS).

### Gas Chromatography-Mass Spectrometry (GC-MS)

Gas chromatography-mass spectrometry (GC-MS) can combine features of gas-chromatography and mass spectrometry to identify different substances within a test sample. In some instances, GC can be used to separate and analyze compounds that can be vaporized without decomposition. Exemplary uses of GC include testing the purity of a particular substance, or separating the different components of a mixture (the relative amounts of such components can also be determined). Mass spectrometry may provide structural identity of the individual components with high molecular specificity and detection sensitivity. In some embodiments, metabolite profiling of the microbiome is determined using gas chromatography-mass spectrometry (GC-MS)

Liquid Chromatography with Nuclear Magnetic Resonance Spectroscopy (LC-NMR)

Liquid Chromatography with Nuclear Magnetic Resonance Spectroscopy (LC-NMR) may refer to a method in which a sample is separated by high-performance liquid chromatography, and then the energy states of spin-active nuclei in the sample, placed in a static magnetic field, are interrogated by inducing transitions between the states via radio frequency irradiation. In some embodiments, metabolite profiling of the microbiome is determined using liquid Chromatography with Nuclear Magnetic Resonance Spectroscopy (LC-NMR).

### Nuclear Magnetic Resonance Spectroscopy (NMR)

Nuclear magnetic resonance spectroscopy (NMR) can exploit magnetic properties of certain atomic nuclei. In some instances, NMR determines the physical and chemical properties of atoms or the molecules in which they are contained. It may rely on the phenomenon of nuclear magnetic resonance and can provide detailed information about the structure, dynamics, reaction state, and chemical environment of molecules. The intramolecular magnetic field around an atom in a molecule can change the resonance frequency, and can give access to details of the electronic structure of a molecule and its individual functional groups. In some embodiments, metabolite profiling of the microbiome is determined using nuclear magnetic resonance spectroscopy (NMR).

### Algorithm-Based Methods

The present disclosure provides algorithm-based methods for building a microbiome metabolic profile of a subject. Non-limiting examples of algorithms that can be used with the disclosure include elastic networks, random forests, support vector machines, and logistic regression.

The algorithms can transform the underlying measurements into a quantitative score or probability relating to, for example, disease risk, disease likelihood, presence or absence of disease, presence or absence of a microbe, treatment response, and/or classification of disease status. The algorithms can aid in the selection of important microbes.

### Analysis

A metabolic profile of a subject can be analyzed to determine information related to the health status of the subject. The information can include, for example, degree of likelihood of a disorder, presence or absence of a disease state, a poor clinical outcome (e.g., no or detrimental response to a therapy or intervention), good clinical outcome (e.g., an improvement in a treated condition in response to a therapy or intervention), elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management.

The analysis can be performed as a part of a diagnostic assay to predict disease status of a subject or likelihood of a subject's response to a therapeutic. The diagnostic assay can use the quantitative score calculated by the algorithms-based methods described herein to perform the analysis.

In some embodiments, an increase in one or more microbes' threshold values or quantitative scores in a subject's microbiome profile is indicative of an increased likelihood of one or more of: a poor clinical outcome, good clinical outcome, elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management.

In some embodiments, a decrease in one or more microbes' threshold values or quantitative scores in a subject's microbiome profile is indicative of a decreased likelihood of one or more of: a poor clinical outcome, good clinical outcome, elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management.

In some embodiments, a decrease in one or more microbes' threshold values or quantitative scores in a subject's microbiome profile is indicative of an increased likelihood of one or more of: a poor clinical outcome, good clinical outcome, elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management.

In some embodiments, an increase in one or more microbes' threshold values or quantitative scores in a subject's microbiome profile is indicative of an decreased likelihood of one or more of: a poor clinical outcome, good clinical outcome, elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management.

In some embodiments, a similar metabolic profile to a reference profile is indicative of an increased likelihood of one or more of: a poor clinical outcome, good clinical outcome, elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management. In some embodiments, a dissimilar microbiome profile to a reference profile is indicative of an increased likelihood of one or more of: a poor clinical outcome, good clinical outcome, elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management.

In some embodiments, a similar microbiome metabolic profile to a reference profile is indicative of a decreased likelihood of one or more of: a poor clinical outcome, good clinical outcome, elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management. In some embodiments, a dissimilar microbiome metabolic profile to a reference profile is indicative of an decreased likelihood of one or more of: a poor clinical outcome, good clinical outcome, elevated or high risk of disease (e.g., compared to a general population), decreased or low risk of disease (e.g., compared to a general population), recurrence, relapse, prognosis, life expectancy, complete response, partial response, stable disease, non-response, and recommended treatments for disease management.

### Accuracy and Sensitivity

The methods provided herein can provide strain classification of a genera, species, or sub-strain level of one or more microbes in a sample with an accuracy of greater than 1%, 5%, 10%, 20%, 25%, 30%, 35%, 40%, 45%, 50%, 55%, 60%, 65%, 70%, 75%, 80%, 85%, 90%, 95%, 96%, 97%, 98%, 99%, 99.2%, 99.5%, 99.6%, 99.7%, 99.8%, or 99.9%. The methods provided herein can provide strain quantification of a genera, species, or sub-strain level of one or more microbes in a sample with an accuracy of greater than 1%, 5%, 10%, 20%, 25%, 30%, 35%, 40%, 45%, 50%, 55%, 60%, 65%, 70%, 75%, 80%, 85%, 90%, 95%, 96%, 97%, 98%, 99%, 99.2%, 99.5%, 99.6%, 99.7%, 99.8%, or 99.9%.

A microbial profiling method of the present disclosure can have an accuracy of 70% or greater based on measurement of 15 or fewer microbes in the biological sample. A profiling method of the present disclosure can have at least an accuracy greater than 70% based on measurement of no more than 2 microbes, 3 or fewer microbes, 4 or fewer microbes, 5 or fewer microbes, 6 or fewer microbes, 7 or fewer microbes, 8 or fewer microbes, 9 or fewer microbes, 10 or fewer microbes, 11 or fewer microbes, no more than 12 microbes, 13 or fewer microbes, 14 or fewer microbes, 15 or fewer microbes, 16 or fewer microbes, 18 or fewer microbes, 19 or fewer microbes, 20 or fewer microbes, 25 or fewer microbes, 30 or fewer microbes, 35 or fewer microbes, 40 or fewer microbes, 45 or fewer microbes, 50 or fewer microbes, 55 or fewer microbes, 60 or fewer microbes, 65 or fewer microbes, 70 or fewer microbes, 75 or fewer microbes, 80 or fewer microbes, 85 or fewer microbes, 90 or fewer microbes, or 100 or fewer microbes, 200 or fewer microbes, 300 or fewer microbes, 400 or fewer microbes, 500 or fewer microbes, 600 or fewer microbes, 700 or fewer microbes, or 800 or fewer microbes.

Diagnostic methods of the present disclosure for a disease or disorder can have at least one of a sensitivity of 70% or greater and specificity of greater than 70% based on measurement of 15 or fewer microbes in the biological sample. Such diagnostic method can have at least one of a sensitivity greater than 70% and specificity greater than 70% based on measurement of no more than 2 microbes, 3 or fewer microbes, 4 or fewer microbes, 5 or fewer microbes, 6 or fewer microbes, 7 or fewer microbes, 8 or fewer microbes, 9 or fewer microbes, 10 or fewer microbes, 11 or fewer microbes, no more than 12 microbes, 13 or fewer microbes, 14 or fewer microbes, 15 or fewer microbes, 16 or fewer microbes, 18 or fewer microbes, 19 or fewer microbes, 20 or fewer microbes, 25 or fewer microbes, 30 or fewer microbes, 35 or fewer microbes, 40 or fewer microbes, 45 or fewer microbes, 50 or fewer microbes, 55 or fewer microbes, 60 or fewer microbes, 65 or fewer microbes, 70 or fewer microbes, 75 or fewer microbes, 80 or fewer microbes, 85 or fewer microbes, 90 or fewer microbes, or 100 or fewer microbes, 200 or fewer microbes, 300 or fewer microbes, 400 or fewer microbes, 500 or fewer microbes, 600 or fewer microbes, 700 or fewer microbes or 800 or fewer microbes.

The methods provided herein can determine a health status of a subject with a specificity greater than 1%, 5%, 10%, 20%, 25%, 30%, 35%, 40%, 45%, 50%, 55%, 60%, 65%, 70%, 75%, 80%, 85%, 90%, 95%, 96%, 97%, 98%, 99%, 99.2%, 99.5%, 99.6%, 99.7%, 99.8%, or 99.9% as determined using a receiver operating characteristic (ROC). The methods provided herein can determine a health status of a subject with a sensitivity greater than 1%, 5%, 10%, 20%, 25%, 30%, 35%, 40%, 45%, 50%, 55%, 60%, 65%, 70%, 75%, 80%, 85%, 90%, 95%, 96%, 97%, 98%, 99%, 99.2%, 99.5%, 99.6%, 99.7%, 99.8%, or 99.9% as determined using an ROC. The methods provided herein can determine a health status of a subject with an area under the curve (AUC) of greater than 0.50, 0.55, 0.60, 0.65, 0.70, 0.75, 0.80, 0.85, 0.90, 0.95, 0.96, 0.97, 0.98, 0.99, 0.992, 0.995, 0.996, 0.997, 0.998, or 0.999 as determined using an ROC.

### Microbiome Associated Disorders

In some embodiments, the disorder is associated with and/or caused by an altered microbiome of the subject. In some embodiments, a disorder is associated with and/or caused by gut dysbiosis. In some embodiments, the disorder is associated with and/or caused by an altered production of one or more short chain fatty acids (SCFAs) in the subject. In some embodiments, the short chain fatty acid is butyrate. In some embodiments, the short chain fatty acid is propionate. In some embodiments, the short chain fatty acid is acetate. In some embodiments, the disorder is caused by reduced butyrate production. For example, a patient can have reduced short-chain fatty acid producing (e.g., butyrate-producing) microbes. Altered SCFA production can be caused by, for example, an altered SCFA pathway (e.g., altered butyrate pathway), altered SCFA-producing microbes, or an increase or decrease in substrate or cofactors needed for the SCFA pathway or SCFA-producing microbes. Altered butyrate production can affect one or more downstream signaling pathways in a subject, which can lead to a disorder. Methods and compositions, for example, comprising probiotics to increase butyrate production, can be used for treating a disorder.

A subject can have a microbiome profile that is a signature or characteristic of a disorder (e.g., a microbiome signature of a disorder). For example, a patient with a metabolic disorder such as IBD or Crohn's disease can have a reduced population of microbes such as bacteriodes, eubacterium, faecalibacterium, and ruminococcus, and/or an increased population of actinomyces and Bifidobacterium. The patient can have reduced butyric acid concentration (e.g., in feces) compared with healthy or reference controls. The microbiota signature of a disorder can be used as a diagnostic for determining a disorder. Imbalance in intestinal microflora constitution can be involved in the pathogenesis of inflammatory bowel disease.

A disorder or condition treated by a composition of the present disclosure can include skin or dermatological disorders, metabolic disorders, neurological disorders, cancer, cardiovascular disorders, immune function disorders, inflammatory disorder, pulmonary disorder, metastasis, a chemotherapy or radiotherapy-induced condition, age-related disorder, a premature aging disorder, and sleep disorders.

Alterations in gut microbiota can be implicated in the pathophysiology of a disorder, for example, skin or dermatological disorders, metabolic disorders, neurological disorders, cancer, cardiovascular disorders, immune function disorders, inflammatory disorder, pulmonary disorder, metastasis, a chemotherapy or radiotherapy-induced condition, age-related disorder, a premature aging disorder, and sleep disorders.

A subject with a metabolic disorder or metabolic syndrome can suffer from comorbidities including, for example, skin or dermatological disorders, metabolic disorders, neurological disorders, cancer, cardiovascular disorders, immune function disorders, inflammatory disorder, pulmonary disorder, metastasis, a chemotherapy or radiotherapy-induced condition, age-related disorder, a premature aging disorder, and sleep disorders.

### Metabolic Disorders

In some embodiments, the disorder is a metabolic disorder. Non-limiting examples of metabolic disorders include diabetes, Type I diabetes mellitus, Type II diabetes mellitus, metabolic syndrome, inflammatory bowel disease, obesity, gestational diabetes, ischemia-reperfusion injury such as hepatic ischemia-reperfusion injury, fatty liver disease such as non-alcoholic fatty liver disease, non-alcoholic steatohepatitis, Crohn's disease, colitis, ulcerative colitis, Pseudomembranous colitis, renal dysfunction, nephrological pathology, and glomerular disease.

Patients with metabolic disorders can have reduced butyrate producers. A subject with a metabolic condition (e.g., Crohn's Disease; inflammatory bowel disease) can show a decrease in Bacteroides, Eubacterium, Faecalibacterium and Ruminococcus; and an increase in Actinomyces and Bifidobacterium; a decrease in butyrate production pathway; a decrease in butyrate producing strains; a decrease in butyric acid concentration (e.g., in feces); and imbalance in intestinal microflora constitution.

In some embodiments, the disorder is Type I diabetes mellitus (T1DM). Patients with T1DM can have reduced bacterial diversity and reduced butyrate producing microbes. Increasing butyrate production, for example by administering a composition comprising A. muciniphila, can be used for T1DM treatment.

In some embodiments, the disorder is inflammatory bowel disease (IBD). Patients with IBD can have reduced butyrate production (e.g., due to reduced butyrate-producing microbes). Increasing butyrate production can result in decreased IBD. Butyrate can ameliorate colonic inflammation associated with IBD.

In some embodiments, the disorder is Crohn's disease. Butyrate can, for example, decrease cytokine (e.g., Tumor Necrosis Factor; proinflammatory cytokine mPRA) production; abolish lipopolysaccharide induced expression of cytokines; and abolish transmigration of NFkappaB (NF-kB) to the nucleus in blood cells. Butyrate can decrease proinflammatory cytokine expression, for example, via inhibition of NF-kB activation and IkappaBalpha (IdBa) degradation. Butyrate can inhibit inflammatory responses (e.g., in Crohn's disease) through NF kappa B inhibition.

In some embodiments, the disorder is non-alcoholic fatty liver disease (NAFLD). Subjects with NAFLD can have reduced butyrate production and/or butyrate-producing microbes. Administration of butyrate-producing microbes (e.g., C. butyricum) can reduce NAFLD progression, reduce hepatic lipid deposition, improve triglyceride content, improve insulin resistance, improve serum endotoxin levels, and improve hepatic inflammatory indexes. Altered gut microbiome can independently cause obesity, which can be an important risk factor for NAFLD. This capability can be attributed to short-chain fatty acids (SCFAs), which are gut microbial fermentation products. SCFAs can account for a large portion of caloric intake of the host. SCFAs can enhance intestinal absorption by activating GLP-2 signaling. Elevated SCFAs can be an adaptive measure to suppress colitis, which could be a higher priority than imbalanced calorie intake. The microbiome of non-alcoholic steatohepatitis (NASH) patients can feature an elevated capacity for alcohol production. The pathomechanisms for alcoholic steatohepatitis can apply to NASH. NAFLD/NASH can be associated with elevated gram-negative microbiome and endotoxemia. NASH patients can exhibit normal serum endotoxin indicating that endotoxemia may not be required for the pathogenesis of NASH. Microbial compositions of the present disclosure can benefit NAFLD/NASH patients.

In some embodiments, the disorder is total hepatic ischemia reperfusion injury. Butyrate preconditioning can improve hepatic function and histology following ischemia-reperfusion injury. Inlammatory factors levels, macrophages activation, TLR4 expression and neutrophil infiltration can be attenduated by butyrate.

In some embodiments, the disorder is gestational diabetes.

### Neurological and Behavioral Conditions

In some embodiments, the disorder is a neurological condition. Neurological conditions include, but are not limited to, neural activity disorders, anxiety, depression, chronic fatigue syndrome, autism, Parkinson's disease, Alzheimer's disease, dementia, amyotrophic lateral sclerosis (ALS), bulbar palsy, pseudobulbar palsy, primary lateral sclerosis, motor neuron dysfunction (MND), mild cognitive impairment (MCI), Huntington's disease, ocular diseases, age-related macular degeneration, glaucoma, vision loss, presbyopia, cataracts, progressive muscular atrophy, lower motor neuron disease, spinal muscular atrophy (SMA), Werdnig-Hoffman Disease (SMA1), SMA2, Kugelberg-Welander Disease (SM3), Kennedy's disease, post-polio syndrome, and hereditary spastic paraplegia. In some embodiments, the disorder is a behavioral condition.

Gut microbes can have a significant impact on nervous system and host behavior. Increasing SCFA production (e.g., by increasing butyrate producers) can, for example, improve brain development, motor activity, reduce anxiety, improve depression, increased immunoregulatory T (Treg) cells, and improved psychological states.

Butyrate can activate intestinal gluconeogenesis in insulin-sensitive and insulin-insensitive states, which can promote glucose and energy homeostasis. Microbial compositions can alter activity in brain regions that control central processing of emotion and sensation.

In some embodiments, methods and compositions of the present disclosure modulate (e.g., reduce) appetite in a subject. In some embodiments, methods and compositions of the present disclosure modulate (e.g., improve) behavior of a subject.

Butyrate production by gut microbiome can decrease appetite, for example, via gut-brain connection. Obese subjects can have increased scores on food addiction and food craving scales when compared to lean subjects. Alterations in gut microbiota can be implicated in the pathophysiology of several brain disorders, including anxiety, depression, and appetite. When fiber is ingested, gut microbes can metabolize the fiber into short chain fatty acids (SFCAs), including butyrate. Butyrate can bind to receptors, for example, G-protein coupled receptors. For example, butyrate can bind to G-protein coupled receptor GPR41 and trigger peptide tyrosine-tyrosine (PYY) and glucagon-like peptide 1 (GLP-1). PYY and GLP-1 can bind to receptors in the enteric nervous system, resulting in signaling to the brain via the vagus nerve that can result in reducing appetite.

In some embodiments, methods of the present disclosure provide a synbiotic (e.g., comprising prebiotics and probiotics) intervention method, which can target a specific gut microbiome biochemical pathway linked to altered brain function and behavior. In some embodiments, methods of the present disclosure provide companion diagnostic for assessing efficacy of microbiome-based treatments of comorbid psychiatric disorders. In some embodiments, methods of the present disclosure provide extension of Boolean implications and application of co-inertia analysis as state-of-the-art statistical methods for exploratory data analysis and biomarker discovery.

Methods and compositions of the present disclosure (e.g., a butyrate producing composition) can alter levels of neurotransmitters substance (e.g., serotonin, dopamine, GABA), neuroactive metabolite (e.g., branched chain and aromatic amino acids, p cresol, N acetyl putrescine, o cresol, phenol sulfate, kinurate, caproate, histamine, agmatine), and inflammatory agents (e.g., lipopolysaccharide, IL-1,IL-6, IL-8, TNF-alpha, CRP) in a subject.

### Immune System Conditions

In some embodiments, the disorder is an immune system disorder. In some embodiments, the disorder is an inflammatory condition.

Non-limiting examples of immune system related disorders include allergies, inflammation, anaphylactic shock, autoimmune diseases, rheumatoid arthritis, systemic lupus erythematosus (SLE), scleroderma, diabetes, Autoimmune enteropathy, Coeliac disease, Crohn's disease, Microscopic colitis, ulcerative colitis, osteoarthritis, osteoporosis, oral mucositis, inflammatory bowel disease, kyphosis, herniated intervertebral disc, ulcerative asthma, renal fibrosis, liver fibrosis, pancreatic fibrosis, cardiac fibrosis, skin wound healing, and oral submucous fibrosis.

In some embodiments, the present disclosure provides methods for treating or reducing the likelihood of conditions resulting from a host immune response to an organ transplant in a subject in need thereof. Non-limiting examples of an organ transplant include a kidney organ transplant, a bone marrow transplant, a liver transplant, a lung transplant, and a heart transplant. In some embodiments, the present disclosure provides methods for treating graft-vs-host disease in a subject in need thereof.

Microbial metabolites can play a role in development of the immune system. Gut microbiome can play a role in the development of allergies. Microbes can mediate immunomodulation. Based on the immunomodulating capacities of bacteria, probiotics can be used for treating eczema, for example, Bifidobacterium bifidum, Bifidobacterium animalis subsp. Lactis, and Lactococcus lactis. Lower amounts of metabolites, SCFAs, succinate, phenylalanine, and alanine can be found in fecal samples of subjects (e.g., children) later developing skin disorders (e.g, eczema), whereas the amounts of glucose, galactose, lactate and lactose can be higher compared to subjects not developing skin disorders. Supplementation of multispecies probiotics can induce higher levels of lactate and SCFAs, and lower levels of lactose and succinate.

Administration of compositions comprising SCFA or SCFA-producing microbes can increase immunoregulatory cells.

### Skin Disorders

In some embodiments, the disorder is a dermatological disorder. Dermatological conditions include, but are not limited to, psoriasis, eczema, rhytides, pruritis, dysesthesia, papulosquamous disorders, erythroderma, lichen planus, lichenoid dermatosis, atopic dermatitis, eczematous eruptions, eosinophilic dermatosis, reactive neutrophilic dermatosis, pemphigus, pemphigoid, immunobullous dermatosis, fibrohistocytic proliferations of skin, cutaneous lymphomas, and cutaneous lupus.

In some embodiments, the disorder is atopic dermatitis. In some embodiments, the disorder is eczema.

Patients with skin disorders (e.g., atopic dermatitis) can have, for example, reduced butyrate producing microbes, lower diversity of the phylum Bacteriodetes, altered diversity of gut microbiome, and altered abundance of C. eutactus.

### Cardiovascular Conditions

In some embodiments, the disorder is a cardiovascular disorder. Non-limiting examples of cardiovascular conditions, include, but are not limited to angina, arrhythmia, atherosclerosis, cardiomyopathy, congestive heart failure, coronary artery disease (CAD), carotid artery disease, endocarditis, heart attack, coronary thrombosis, myocardial infarction (MI), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease, peripheral artery disease (PAD), cardiac stress resistance, and stroke.

### Pulmonary Conditions

In some embodiments, the disorder is a pulmonary condition. Pulmonary conditions include, but are not limited to, idiopathic pulmonary fibrosis (IPF), chronic obstructive pulmonary disease (COPD), asthma, cystic fibrosis, bronchiectasis, and emphysema.

In some embodiments, the subject has been exposed to environmental pollutants, for example, silica. A subject can be exposed to an occupational pollutant, for example, dust, smoke, asbestos, or fumes. In some embodiments, the subject has smoked cigarettes.

In some embodiments, the subject has a connective tissue disease. The connective tissue disease can be, for example, rheumatoid arthritis, systemic lupus erythematosus, scleroderma, sarcoidosis, or Wegener's granulomatosis. In some embodiments, the subject has an infection. In some embodiments, the subject has taken or is taking medication or has received radiation therapy to the chest. The medication can be, for example, amiodarone, bleomycin, busufan, methotrexate, or nitrofurantoin.

### Cancer

In some embodiments, the disorder is cancer. Non-limiting examples of cancers include: acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unkown primary site, urethral cancer, uterine sarcoma, vaginal cancer, vulvar cancer, Waldenström macroglobulinemia, and Wilms tumor.

In some embodiments, the disorder is colorectal cancer.

Subjects with cancer can have altered butyrate production, for example, due to reduced butyrate-producing microbes. Methods and compositions of the present disclosure can be used for tumor treatment and reduction, for example, by delivering butyrate producing microbes to the subject.

Most cell types in the body can utilize glucose as their primary energy source, while normal colonocytes can rely on butyrate for about 60-70% of their energy. Butyrate can undergo beta-oxidation in the mitochondria, which can support energy homeostasis for rapid cell proliferation of the colonic epithelium. In contrast, tumor cells (e.g., colorectal tumor cells) can switch to glucose utilization and aerobic glycolysis. As a result of this metabolic shift, butyrate may not metabolize in the mitochondria of tumor cells to the same extent and can accumulate in the nucleus. In the nucleus, butyrate can function as a histone deacetylase (HDAC) inhibitor to epigenetically regulate gene expression. Patients with colitis can have, for example, up to a 10-fold increase of colorectal cancer.

Methods and compositions of the present disclosure can increase levels of butyrate, which can serve as an endogenous HDAC inhibitor. Since bioavailability of butyrate can be primarily restricted to the colon, butyrate may not have adverse effects associated with synthetic HDAC inhibitors such as those used in chemotherapy. Butyrate can target tumor cells, for example, because of the Warburg effect.

Dietary risk of cancer (e.g., colon cancer) can be mediated by dysbiosis of gut microbiota and their metabolites (e.g., SCFAs such as butyrate). Dietary fiber and/or complex carbohydrates can promote saccharolytic fermentation, which can yield anti-inflammatory and antiproliferative SCFAs such as butyrate. Consumption of red meat can generate inflammatory and genotoxic metabolites by promoting proteolytic fermentation, hydrogen sulfide production from the sulfur-rich amino acid content of red meat, and expose colonic mucosa to carcinogenic constituents.

Dietary fiber intake can promote a healthy gut microbiome, which in turn can enhance SCFA (e.g., butyrate, acetate, propionate) production. Enhanced SCFA production can result in, for example, reduced food intake, increased energy levels, better colon health, promote healthy gut intestinal barrier, reduce colon content transit time and exposure to carcinogens, cancer cell cycle arrest and apoptosis, inhibition of cancer cell migration and invasion, inhibition of early colon lesion, inhibition of adenoma formation, inhibition of colon adenoma, inhibition of tumor progression, and inhibition of colon carcinoma.

### Microbial Compositions

Methods and compositions of the present disclosure can modulate and/or restore SCFA production (e.g., butyrate production) in a subject. For example, the SCFA (e.g., butyrate) production can be increased in a subject. The butyrate production can be increased, for example, by at least about: 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, 5%, 10%, 15%, 20%, 25%, 30%, 35%, 40%, 45%, 50%, 55%, 60%, 65%, 70%, 75%, 80%, 85%, 90%, 95%, or 100%. The butyrate production can be decreased, for example, by at least about: 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, 5%, 10%, 15%, 20%, 25%, 30%, 35%, 40%, 45%, 50%, 55%, 60%, 65%, 70%, 75%, 80%, 85%, 90%, 95%, or 100%.

Methods and compositions of the present disclosure can be used to modulate the weight of a subject. The weight can be increased or decreased. A subject can lose or gain at least about: 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, 5%, 10%, 15%, 20%, 25%, 30%, 35%, 40%, 45%, or 50% of the body weight.

A therapeutic or strain consortia can comprise one or more microorganisms selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, and any combination thereof.

A therapeutic or strain consortia can comprise one or more microorganisms selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, and any combination thereof.

A therapeutic or strain consortia can comprise microorganisms from a phylum selected from one or more of: Actinobacteria, Bacteroidetes, Cyanobacteria, Firmicutes, Fusobacteria, Proteobacteria, Spirochaetes, Tenericutes, or Verrucomicrobia.

A therapeutic or strain consortia can comprise microorganisms from a family selected from one or more of: Alcaligenaceae, Bifidobacteriaceae, Bacteroidaceae, Clostridiaceae, Coriobacteriaceae, Enterobacteriaceae, Enterococcaceae, Erysipelotricaceae, Eubacteriaceae, Incertae-Cedis-XIII, Incertae-Sedis-XIV, Lachnospiraceae, Lactobacillaceae, Pasturellaceae, Peptostreptococcaceae, Porphyromonadaceae, Prevotellaceae, Rikenellaceae, Ruminococcaceae, Streptococcaceae, Veillonellaceae, or Verrucomicrobiaceae.

A therapeutic or strain consortia can comprise one or more microorganisms with at least about: 70%, 75%, 80%, 85%, 87%, 90%, 91%, 92%, 93%, 94%, 95%, 96%, 97%, 98%, 99%, 99.5%, or 100% sequence identity to the rRNA (e.g., 16SrRNA and/or 23S rRNA) of a microorganism selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, and any combination thereof.

A microbial composition can comprise a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, and any combination thereof.

In some embodiments, provided are compositions to treat a disorder comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, and any combination thereof.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from an Lactobacillus species.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from an Akkermansia.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Bifidobacterium.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Clostridium.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Eubacterium,

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Verrucomicrobium,

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Firmicute.

In some embodiments, provided are pharmaceutical microbial compositions comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Lactobacillus reuteri (e.g., Lactobacillus reuteri RC-14, Lactobacillus reuteri L22), Streptococcus mutans, Stenotrophomonas nitritireducens, and any combination thereof.

In some embodiments, provided are pharmaceutical microbial compositions comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Lactobacillus rhamnosus, Faecalibacterium prausnitzii, Oscillospira guilliermondii, Clostridium orbiscindens, Clostridium colinum, Clostridium aminophilum, Ruminococcus obeum, and any combination thereof.

In some embodiments, provided are pharmaceutical microbial compositions comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and any combination thereof.

In some embodiments, provided are pharmaceutical microbial compositions comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, Faecalibacterium prausnitzii, and any combination thereof.

In some embodiments, provided are pharmaceutical microbial compositions comprising a therapeutically-effective amount of a population of isolated and purified microbes, wherein the population of isolated and purified microbes comprises a microbe with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Clostridium beijerinckii, Clostridium butyricum, Eubacterium hallii, and any combination thereof.

In some embodiments, provided are pharmaceutical microbial compositions comprising a therapeutically-effective amount of a population of isolated and purified microbes, wherein said population of isolated and purified microbes comprises at least 2, 3, 4, 5, 6, 7, 8, 9, 10, 11, 12, 13, 14, or 15 different microbes strains or species, wherein each microbial strain comprises a rRNA sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence of a microbe selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Acidaminococcus fermentans, Acidaminococcus intestine, Blautia hydrogenotrophica, Citrobacter amalonaticus, Citrobacter freundii, Clostridium aminobutyricum Clostridium bartlettii, Clostridium cochlearium, Clostridium kluyveri, Clostridium limosum, Clostridium malenominatum, Clostridium pasteurianum, Clostridium peptidivorans, Clostridium saccharobutylicum, Clostridium sporosphaeroides, Clostridium sticklandii, Clostridium subterminale, Clostridium symbiosum, Clostridium tetanomorphum, Eubacterium oxidoreducens, Eubacterium pyruvativorans, Methanobrevibacter smithii, Morganella morganii, Peptoniphilus asaccharolyticus, Peptostreptococcus, and any combination thereof.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Akkermansia muciniphila.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Anaerostipes caccae.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Bifidobacterium adolescentis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Bifidobacterium bifidum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Bifidobacterium infantis

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Bifidobacterium longum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Butyrivibrio fibrisolvens.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium acetobutylicum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium aminophilum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium beijerinckii.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium butyricum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium colinum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium coccoides.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium indolis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium nexile.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium orbiscindens.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium propionicum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium xylanolyticum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Enterococcus faecium.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium hallii.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium rectale.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Faecalibacterium prausnitzii.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Fibrobacter succinogenes.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus acidophilus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus brevis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus bulgaricus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus casei.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus caucasicus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus fermentum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus helveticus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus lactis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus plantarum

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus reuteri.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus rhamnosus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Oscillospira guilliermondii.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia cecicola.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia inulinivorans.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Ruminococcus flavefaciens.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Ruminococcus gnavus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Ruminococcus obeum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Stenotrophomonas nitritireducens.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus cremoris.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus faecium.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus infantis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus mutans.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus thermophilus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Anaerofustis stercorihominis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Anaerostipes hadrus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Anaerotruncus colihominis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium sporogenes.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium tetani.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Coprococcus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Coprococcus eutactus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium cylindroides.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium dolichum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium ventriosum.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia faeccis

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia hominis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia intestinalis.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a vinegar-producing microbe.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus bifidus.

In one embodiment, a composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus johnsonii

A therapeutic composition can comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, or at least 50, or at least 75, or at least 100 different microbes (e.g, strains, species, phyla, classes, orders, families, or genuses of microbes). A therapeutic composition can comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 45, or at most 50, or at most 75, or at most 100 different microbes (e.g., strains, species, phyla, classes, orders, families, or genuses of microbes).

In some embodiments, combining one or more microbes in a therapeutic composition or consortia increases or maintains the stability of the microbes in the composition compared with the stability of the microbes alone. A therapeutic consortium of microbes can provide a synergistic stability compared with the individual strains.

In some embodiments, combining one or more microbes in a therapeutic composition or consortia can provide a synergistic effect when administered to the individual. For example, administration of a first microbe may be beneficial to a subject and administration of a second microbe may be beneficial to a subject but when the two microbes are administered together to a subject, the benefit is greater than the either benefit alone.

Different types of microbes in a therapeutic composition can be present in the same amount or in different amounts. For example, the ratio of two bacteria in a therapeutic composition can be about 1:1, 1:2, 1:5, 1:10, 1:25, 1:50, 1:100, 1:1000, 1:10,000, or 1:100,000.

Compositions of the disclosure can include one or more Lactobacillus species. Non-limiting examples of lactobacillus species include, for example, L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviarius, L. bifermentans, L. bifidus, L. brevis, L. buchneri, L. bulgaricus, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. bulgaricus, L. delbrueckii subsp. delbrueckii, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. hammesii, L. hamsteri, L. harbinensis, L. hayakitensis, L. helveticus, L. hilgardii, L. homohiochii, L. finers, L. ingluviei, L. intestinalis, L. jensenii, L. johnsonii, L. kalixensis, L. kefiranofaciens, L. kefiri, L. kimchii, L. kitasatonis, L. kunkeei, L. leichmannii, L. lindneri, L. malefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. paralimentarius, L. paraplantarum, L. pentosus, L. perolens, L. plantarum, L. pontis, L. protectus, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae, and L. zymae.

The compositions can include metabolites for example, to assist in the initial efficacy of the therapeutic before the microbes can produce their own metabolites. Metabolites can include short-chain fatty acids (SCFAs), which can be a subgroup of fatty acids with 6 or less carbons in their aliphatic tails, for example, acetate, propionate, isobutyrate, isovaleric acid, 3-methylbutanoic acid, valeric acid, pentanoic acid, delphinic acid, isopentanoic acid, and butyrate.

The composition can include one or more prebiotics. In one non-limiting example, the prebiotic is an oligosaccharide.

In some embodiments, the prebiotic and probiotic consortia are chosen to create an entirely self-sufficient system that does not require any external input. A combination of probiotics and prebiotics can provide a complete system for producing amino acids, polyphenols, vitamins, and other compounds of nutritive value in a subject. A subject can be treated with a combination of SCFA-producing probiotics and prebiotics comprising dietary fiber and other agents required for the activity of the SCFA-producing probiotics. In this manner, the prebiotic and probiotic form a self-sufficient system, wherein the probiotic converts the prebiotic dietary fiber to SCFAs (butyrate, acetate, and/or propionate), which can trigger downstream signaling for controlling a disorder in the subject.

In some embodiments, a composition comprises a therapeutically-effective amount of an isolated and/or purified microbe with a butyrate kinase sequence (e.g., amino acid or nucleotide sequence) comprising at least about: 85%, 87%, 90%, 92%, 95%, 96%, 97%, 98%, 99%, 99.5%, or 100% sequence identity to butyrate kinase. The sequence (e.g., amino acid or nucleotide sequence) can comprise at least about: 85%, 87%, 90%, 92%, 95%, 96%, 97%, 98%, 99%, 99.5%, or 100% sequence identity to, for example, butyrate kinase (e.g., EC 2.7.2.7; MetaCyc Reaction ID R11-RXN).

In some embodiments, a composition comprises a therapeutically-effective amount of an isolated and/or purified microbe with a butyrate-coenzyme A sequence (e.g., amino acid or nucleotide sequence) comprising at least about: 85%, 87%, 90%, 92%, 95%, 96%, 97%, 98%, 99%, 99.5%, or 100% sequence identity to a butyrate-coenzyme A.

In some embodiments, a composition comprises a therapeutically-effective amount of an isolated and/or purified microbe with a butyrate-coenzyme A transferase or butyryl-Coenzyme A:acetoacetate CoenzymeA transferase sequence comprising at least about: 85%, 87%, 90%, 92%, 95%, 96%, 97%, 98%, 99%, 99.5%, or 100% sequence identity to the butyrate-coenzyme A transferase sequence. The sequence (e.g., amino acid or nucleotide sequence) can comprise at least about: 85%, 87%, 90%, 92%, 95%, 96%, 97%, 98%, 99%, 99.5%, or 100% sequence identity to, for example, butyryl-Coenzyme A:acetoacetate CoenzymeA transferase (e.g., EC 2.8.3.9; MetaCyc Reaction ID 2.8.3.9-RXN).

In some embodiments, a composition comprises a therapeutically-effective amount of an isolated and/or purified microbe with a acetate Coenzyme A transferase comprising at least about: 85%, 87%, 90%, 92%, 95%, 96%, 97%, 98%, 99%, 99.5%, or 100% sequence identity to acetate Coenzyme A transferase sequence. The sequence (e.g., amino acid or nucleotide sequence) can comprise at least about: 85%, 87%, 90%, 92%, 95%, 96%, 97%, 98%, 99%, 99.5%, or 100% sequence identity to, for example, acetate Coenzyme A transferase (e.g., EC 2.8.3.1/2.8.3.8; MetaCyc Reaction ID BUTYRATE-KINASE-RXN)

In some embodiments, a composition comprises a therapeutically-effective amount of an isolated and/or purified microbe with a protein involved in butyrate-pathway (e.g, butyrate producing enzyme).

### Compositions for Administration to a Subject

Provided herein are compositions that may be administered as therapeutics and/or cosmetics. One or more microorganisms described herein can be used to create a pharmaceutical formulation comprising an effective amount of the composition for treating a subject. The microorganisms can be in any suitable formulation. Some non-limiting examples can include topical, capsule, pill, enema, liquid, injection, and the like. In some embodiments, the one or more strains disclosed herein may be included in a food or beverage product, cosmetic, or nutritional supplement.

A composition of the present disclosure can be a combination of any microorganisms described herein with other components, such as carriers, stabilizers, diluents, dispersing agents, suspending agents, thickening agents, and/or excipients. The composition can facilitate administration of the microorganisms to a subject. Compositions can be administered in therapeutically-effective amounts as compositions by various forms and routes including, for example, oral, topical, rectal, transdermal, mucosal, and vaginal administration. A combination of administration routes can be utilized. The composition can be administered as therapeutics and/or cosmetics.

The composition can be administered by a suitable method to any suitable body part or body surface of the subject, for example, that shows a correlation with a disorder.

In some embodiments, the composition is administered to a part of the gastrointestinal tract of a subject. Non-limiting examples of parts of gastrointestinal tract include oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract. In some embodiments, the composition is administered to multiple body parts or surfaces, for example, skin and gut.

The composition can include one or more active ingredients. Active ingredients can be selected from the group consisting of: metabolites, bacteriocins, enzymes, anti-microbial peptides, antibiotics, prebiotics, probiotics, glycans (as decoys that would limit specific bacterial/viral binding to the intestinal wall), bacteriophages, and microorganisms.

In some embodiments, the formulation comprises a prebiotic. In some embodiments, the prebiotic is inulin. In some embodiments, the prebiotic is a fiber. The prebiotic, for example, inulin can serve as an energy source for the microbial formulation.

The compositions can be administered topically. The compositions can be formulated as a topically administrable composition, such as solutions, suspensions, lotions, gels, pastes, medicated sticks, balms, creams, ointments, liquids, wraps, adhesives, or patches. The compositions can contain solubilizers, stabilizers, tonicity enhancing agents, buffers, and/or preservatives.

The compositions can be administered orally, for example, through a capsule, pill, powder, tablet, gel, or liquid, designed to release the composition in the gastrointestinal tract.

In some embodiments, administration of a formulation occurs by injection, for example, for a formulation comprising, for example, butyrate, propionate, acetate, and short-chain fatty acids (SCFAs). In some embodiments, administration of a formulation occurs by a suppository and/or by enema. In some embodiments, a combination of administration routes is utilized.

Microbial compositions can be formulated as a dietary supplement. Microbial compositions can be incorporated with vitamin supplements. Microbial compositions can be formulated in a chewable form such as a probiotic gummy. Microbial compositions can be incorporated into a form of food and/or drink. Non-limiting examples of food and drinks in which the microbial compositions can be incorporated include, for example, bars, shakes, juices, infant formula, beverages, frozen food products, fermented food products, and cultured dairy products such as yogurt, yogurt drink, cheese, acidophilus drinks, and kefir.

A formulation of the disclosure can be administered as part of a fecal transplant process. A formulation can be administered to a subject by a tube, for example, nasogastric tube, nasojejunal tube, nasoduodenal tube, oral gastric tube, oral jejunal tube, or oral duodenal tube. A formulation can be administered to a subject by colonoscopy, endoscopy, sigmoidoscopy, and/or enema.

In some embodiments, the microbial composition is formulated such that the one or more microbes can replicate once they are delivered to the target habitat (e.g., gut). In some embodiments, the microbial composition is formulated such that the one or more microbes are viable in the target habitat (e.g., gut). In one non-limiting example, the microbial composition is formulated in a pill, such that the pill has a shelf life of at least about: 1, 2, 3, 4, 5, 6, 7, 8, 9, 10, 11, or 12 months. In another non-limiting example, the storage of the microbial composition is formulated so that the microbes can reproduce in the target habitat, e.g, gut. In some embodiments, other components may be added to aid in the shelf life of the microbial composition. In some embodiments, one or more microbes may be formulated in a manner that it is able to survive in a non-natural environment. For example, a microbe that is native to the gut may not survive in an oxygen-rich environment. To overcome this limitation, the microbe may be formulated in a pill that can reduce or eliminate the exposure to oxygen. Other strategies to enhance the shelf life of microbes may include other microbes (e.g., if the bacterial consortia comprises a composition whereby one or more strains is helpful for the survival of one or more strains).

In some embodiments, a microbial composition is lyophilized (e.g., freeze-dried) and formulated as a powder, tablet, enteric-coated capsule (e.g., for delivery to the gut such as ileum and/or colon region), or pill that can be administered to a subject by any suitable route. The lyophilized formulation can be mixed with a saline or other solution prior to administration.

In some embodiments, a microbial composition is formulated for oral administration, for example, as an enteric-coated capsule or pill, for delivery of the contents of the formulation to the ileum and/or colon regions of a subject.

In some embodiments, the microbial composition is formulated for oral administration. In some embodiments, the microbial composition is formulated as an enteric-coated pill or capsule for oral administration. In some embodiments, the microbial composition is formulated for delivery of the microbes to the ileum region of a subject. In some embodiments, the microbial composition is formulated for delivery of the microbes to the colon region (e.g., upper colon) of a subject. In some embodiments, the microbial composition is formulated for delivery of the microbes to the ileum and colon (e.g., upper colon) regions of a subject.

An enteric-coating can protect the contents of a formulation, for example, oral formulation such as pill or capsule, from the acidity of the stomach. An enteric-coating can provide delivery to the ileum and/or upper colon regions. A microbial composition can be formulated such that the contents of the composition may not be released in a body part other than the gut region, for example, ileum and/or colon region of the subject. Non-limiting examples of enteric coatings include pH sensitive polymers (e.g., eudragit FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers. In some embodiments, the enteric coating is formed by a pH sensitive polymer. In some embodiments, the enteric coating is formed by eudragit FS30D.

The enteric coating can be designed to dissolve at any suitable pH. In some embodiments, the enteric coating is designed to dissolve at a pH greater than from about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units. The enteric coating can be designed to dissolve in the gut, for example, ileum and/or colon region. The enteric coating can be designed to not dissolve in the stomach.

The formulation can be stored in cold storage, for example, at a temperature of about −80° C., about −20° C., about −4° C., or about 4° C. Compositions provided herein can be stored at any suitable temperature. The storage temperature can be, for example, about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C. In some embodiments, the storage temperature is between about 2° C. to about 8° C. Storage of microbial compositions at low temperatures, for example from about 2° C. to about 8° C., can keep the microbes alive and increase the efficiency of the composition. The cooling conditions can also provide soothing relief to patients. Storage at freezing temperature, below 0° C., with a cryoprotectant can further extend stability.

A composition of the disclosure can be at any suitable pH. The pH of the composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. The pH of the composition can be, for example, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0 pH units. If the pH is outside the range desired by the formulator, the pH can be adjusted by using sufficient pharmaceutically-acceptable acids and bases. In some embodiments, the pH of the composition is from about 4 to about 6 pH units. In some embodiments, the pH of the composition is about 5.5 pH units.

Microbial compositions can be formulated as a dietary supplement. Microbial compositions can be incorporated with vitamin supplements. Microbial compositions can be formulated in a chewable form such as a probiotic gummy. Microbial compositions can be incorporated into a form of food and/or drink. Non-limiting examples of food and drinks where the microbial compositions can be incorporated include, for example, bars, shakes, juices, infant formula, beverages, frozen food products, fermented food products, and cultured dairy products such as yogurt, yogurt drink, cheese, acidophilus drinks, and kefir.

A composition of the disclosure can be administered as part of a fecal transplant process. A composition can be administered to a subject by a tube, for example, nasogastric tube, nasojejunal tube, nasoduodenal tube, oral gastric tube, oral jejunal tube, or oral duodenal tube. A composition can be administered to a subject by colonoscopy, endoscopy, sigmoidoscopy, and/or enema.

In some embodiments, a microbial composition is lyophilized (freeze-dried) and formulated as a powder, tablet, enteric-coated capsule, or pill that can be administered to a subject by any suitable route, for example, oral, enema, suppository, or injection. The lyophilized composition can be mixed with a saline or other solution prior to administration.

In some embodiments, the administration of a composition of the disclosure can be preceded by, for example, colon cleansing methods such as colon irrigation/hydrotherapy, enema, administration of laxatives, dietary supplements, dietary fiber, enzymes, and magnesium.

In some embodiments, the microbes are formulated as a population of spores. Spore-containing compositions can be administered by any suitable route described herein. Orally administered spore-containing compositions can survive the low pH environment of the stomach. The amount of spores employed can be, for example, from about 1% w/w to about 99% w/w of the entire composition.

Compositions provided herein can include the addition of one or more agents to the therapeutics or cosmetics in order to enhance stability and/or survival of the microbial composition. Non-limiting example of stabilizing agents include genetic elements, glycerin, ascorbic acid, skim milk, lactose, tween, alginate, xanthan gum, carrageenan gum, mannitol, palm oil, and poly-L-lysine (POPL).

In some embodiments, a composition comprises recombinant microbes or microbes that have been geneticallly modified. In some embodiments, the composition comprises microbes that can be regulated, for example, a microbe comprising an operon to control microbial growth.

A composition can be customized for a subject. A custom composition can comprise, for example, a prebiotic, a probiotic, an antibiotic, or a combination of active agents described herein. Data specific to the subject comprising for example age, gender, and weight can be combined with an analysis result to provide a therapeutic agent customized to the subject. For example, a subject's microbiome found to be low in a specific microbe relative to a sub-population of healthy subjects matched for age and gender can be provided with a therapeutic and/or cosmetic composition comprising the specific microbe to match that of the sub-population of healthy subjects having the same age and gender as the subject.

In some embodiments, a composition is administered before, during, and/or after treatment with an antimicrobial agent such as an antibiotic. For example, the composition can be administered at least 1 hour, 2 hours, 5 hours, 12 hours, 1 day, 3 days, 1 week, 2 weeks, 1 month, 6 months, or 1 year before and/or after treatment with an antibiotic. The composition can be administered at most 1 hour, 2 hours, 5 hours, 12 hours, 1 day, 3 days, 1 week, 2 weeks, 1 month, 6 months, or 1 year before and/or after treatment with an antibiotic.

In some embodiments, the formulation is administered after treatment with an antibiotic. For example, the formulation can be administered after the entire antibiotic regimen or course is complete. In some embodiments, the formulation is administered concurrently with an antibiotic.

In some embodiments, a formulation is administered before, during, and/or after food intake by a subject. In some embodiments, the formulation is administered with food intake by the subject. In some embodiments, the formulation is administered with (e.g., simultaneously) with food intake.

In some embodiments, the formulation is administered before food intake by a subject. In some embodiments, the formulation is more effective or potent at treating a microbial condition when administered before food intake. For example, the formulation can be administered about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at least about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at most about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject.

In some embodiments, the formulation is administered after food intake by the subject. In some embodiments, the formulation is more effective or potent at treating a microbial condition when administered after food intake. For example, the formulation can be administered at least about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject. For example, the formulation can be administered at most about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject.

Formulations provided herein can include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

A therapeutic or cosmetic composition can include carriers and excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavouring agents, dispersion enhancer, disintegrant, coating agents, plasticizers, preservatives, suspending agents, emulsifying agents, plant cellulosic material and spheronization agents, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa., 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), each of which is incorporated by reference in its entirety.

A composition can be substantially free of preservatives. In some embodiments, the composition may contain at least one preservative.

A composition can be encapsulated within a suitable vehicle, for example, a liposome, a microspheres, or a microparticle. Microspheres formed of polymers or proteins can be tailored for passage through the gastrointestinal tract directly into the blood stream. Alternatively, the compound can be incorporated and the microspheres, or composite of microspheres, and implanted for slow release over a period of time ranging from days to months.

A composition can be formulated as a sterile solution or suspension. The therapeutic or cosmetic compositions can be sterilized by conventional techniques or may be sterile filtered. The resulting aqueous solutions may be packaged for use as is, or lyophilized. The lyophilized preparation of the microbial composition can be packaged in a suitable form for oral administration, for example, capsule or pill.

The compositions can be administered topically and can be formulated into a variety of topically administrable compositions, such as solutions, suspensions, lotions, gels, pastes, medicated sticks, balms, creams, and ointments. Such compositions can contain solubilizers, stabilizers, tonicity enhancing agents, buffers and preservatives.

The compositions can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, can be used.

Microbial compositions can be formulated using one or more physiologically-acceptable carriers comprising excipients and auxiliaries, which facilitate processing of the microorganisms into preparations that can be used pharmaceutically. Compositions can be modified depending upon the route of administration chosen. Compositions described herein can be manufactured in a conventional manner, for example, by means of conventional mixing, dissolving, granulating, dragee-making, levigating, encapsulating, entrapping, emulsifying or compression processes.

Compositions containing microbes described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Microbial compositions can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The composition can be packed together or separately, in a single package or in a plurality of packages. One or all of the therapeutic agents can be given in multiple doses. If not simultaneous, the timing between the multiple doses may vary to as much as about a month.

Compositions described herein can be administered before, during, or after the occurrence of a disease or condition, and the timing of administering the composition can vary. For example, the microbial composition can be used as a prophylactic and can be administered continuously to subjects with a propensity to conditions or diseases in order to lessen a likelihood of the occurrence of the disease or condition. The microbial compositions can be administered to a subject during or as soon as possible after the onset of the symptoms. The administration of the microbial compositions can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any composition described herein. A microbial composition can be administered as soon as is practicable after the onset of a disease or condition is detected or suspected, and for a length of time necessary for the treatment of the disease, such as, for example, from about 1 month to about 3 months. The length of treatment can vary for each subject.

Compositions of the present disclosure can be administered in combination with another therapy, for example, immunotherapy, chemotherapy, radiotherapy, anti-inflammatory agents, anti-viral agents, anti-microbial agents, and anti-fungal agents.

Compositions of the present disclosure can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

For example, the composition is formulated for administration via pH-dependent release delivery, microbially-triggered delivery, time-controlled delivery, osmotically-regulated delivery, pressure-controlled delivery, multi matrix systems delivery, bioadhesion delivery, or multiparticulate delivery. The composition can also be formulated for release in the small or large intestine, colon, rectum, stomach, anus, or esophagus.

### Dosage

The appropriate quantity of a therapeutic or cosmetic composition to be administered, the number of treatments, and unit dose can vary according to a subject and/or the disease state of the subject.

Compositions described herein can be in unit dosage forms suitable for single administration of precise dosages. In unit dosage form, the formulation can be divided into unit doses containing appropriate quantities of one or more microbial compositions. The unit dosage can be in the form of a package containing discrete quantities of the formulation. Non-limiting examples are liquids in vials or ampoules. Aqueous suspension compositions can be packaged in single-dose non-reclosable containers. The composition can be in a multi-dose format. Multiple-dose reclosable containers can be used, for example, in combination with a preservative. Formulations for parenteral injection can be presented in unit dosage form, for example, in ampoules, or in multi-dose containers with a preservative.

The dosage can be in the form of a solid, semi-solid, or liquid composition. Non-limiting examples of dosage forms suitable for use in the present disclosure include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

A microbe can be present in any suitable concentration in a composition. The concentration of a microbe can be for example, from about 10¹ to about 10¹⁸ colony forming units (CFU). The concentration of a microbe can be, for example, about 10¹, about 10², about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, or about 10¹⁸ CFU. The concentration of a microbe can be, for example, at least about 10¹, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵, at least about 10¹⁶, at least about 10¹⁷, or at least about 10¹⁸ CFU. The concentration of a microbe can be, for example, at most about 10¹, at most about 10², at most about 10³, at most about 10⁴, at most about 10⁵, at most about 10⁶, at most about 10⁷, at most about 10⁸, at most about 10⁹, at most about 10¹⁰, at most about 10¹¹, at most about 10¹², at most about 10¹³, at most about 10¹⁴, at most about 10¹⁵, at most about 10¹⁶, at most about 10¹⁷, or at most about 10¹⁸ CFU. In some embodiments, the concentration of a microbe is from about 10⁸ CFU to about 10⁹ CFU. In some embodiments, the concentration of a microbe is about 10⁸ CFU. In some embodiments, the concentration of a microbe is about 10⁹ CFU. In some embodiments, the concentration of a microbe is about 10¹⁰ CFU. In some embodiments, the concentration of a microbe is at least about 10⁸ CFU. In some embodiments, the concentration of a microbe is at least about 10⁹ CFU.

The concentration of a microbe in a formulation can be equivalent to, for example, about: 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 70, 80, 90, or 100 OD units. The concentration of a microbe in a formulation can be equivalent to, for example, at least about: 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 70, 80, 90, or 100 OD units. The concentration of a microbe in a formulation can be equivalent to, for example, at most about: 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 70, 80, 90, or 100 OD units.

Compositions of the present disclosure can be formulated with any suitable therapeutically-effective concentration of an active ingredient. For example, the therapeutically-effective concentration of a prebiotic can be at least about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 125 mg/mL, about 130 mg/mL, about 140 mg/mL, or about 150 mg/mL. For example, the therapeutically-effective concentration of a prebiotic can be at most about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 125 mg/mL, about 130 mg/mL, about 140 mg/mL, or about 150 mg/mL. For example, the therapeutically-effective concentration of a prebiotic can be about 1 mg/mL, about 2 mg/mL, about 3 mg/mL, about 4 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 125 mg/mL, about 130 mg/mL, about 140 mg/mL, or about 150 mg/mL. In some embodiments, the concentration of a prebiotic in a composition is about 70 mg/mL. In some embodiments, the prebiotic is inulin.

Compositions of the present disclosure can be administered, for example, 1, 2, 3, 4, 5, or more times daily. Compositions of the present disclosure can be administered, for example, daily, every other day, three times a week, twice a week, once a week, or at other appropriate intervals for treatment of the condition. Compositions of the present disclosure can be administered, for example, for 1, 2, 3, 4, 5, 6, 7, or more days. Compositions of the present disclosure can be administered, for example, for 1, 2, 3, 4, 5, 6, 7, or more weeks. Compositions of the present disclosure can be administered, for example, for 1, 2, 3, 4, 5, 6, 7, or more months.

In practicing the methods of treatment or use provided herein, therapeutically-effective amounts of the compounds described herein are administered in compositions to a subject having a disease or condition to be treated. A therapeutically-effective amount can vary widely depending on the severity of the disease, the age and relative health of the subject, the potency of the compounds used, and other factors.

Subjects can be, for example, mammal, humans, pregnant women, elderly adults, adults, adolescents, pre-adolescents, children, toddlers, infants, newborn, or neonates. A subject can be a patient. In some embodiments, a subject is a human. In some embodiments, a subject is a child (i.e., a young human being below the age of puberty). In some embodiments, a subject is an infant. A subject can be an individual enrolled in a clinical study. A subject can be a laboratory animal, for example, a mammal, or a rodent. In some embodiments, the subject is an obese or overweight subject. In some embodiments, the subject is a formula-fed infant.

### Computer Systems

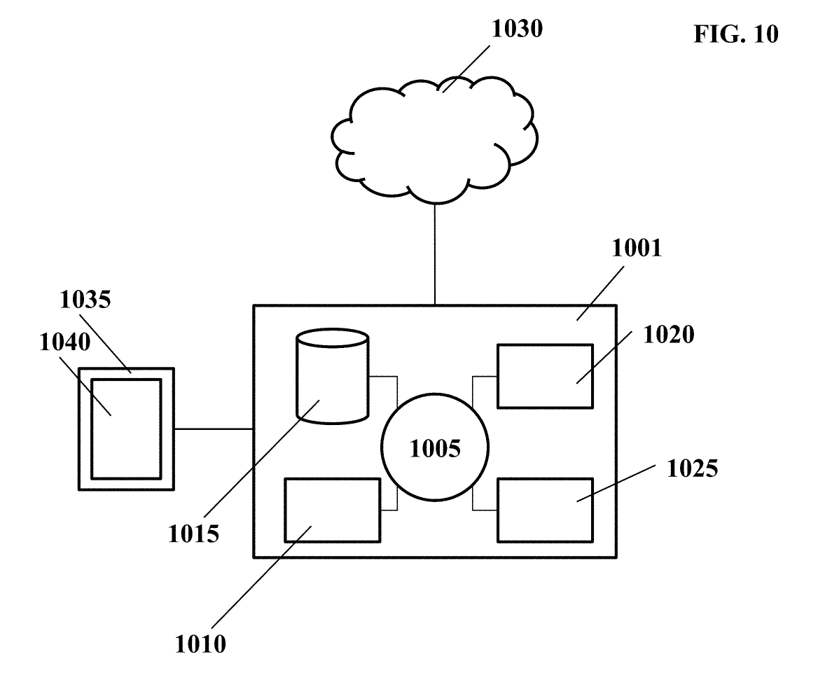

The present disclosure provides computer systems that are programmed to implement methods of the disclosure. FIG. 10 shows a computer system 1001 that is programmed or otherwise configured to implement methods provided herein.

The computer system 1001 can regulate various aspects of the present disclosure, such as, for example, obtaining sequencing information of a sample, identifying organisms or microbes in a population, identifying metabolic pathways or reactions associated with organisms or microbes in a population, identifying presence of metabolic pathways, as indicated by identifying a nucleic acid marker that encodes a component of the metabolic pathway in a genome of the organism, and determining abundance of metabolic pathways. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet.

The network 1030 in some cases is a telecommunication and/or data network. The network 1030 can include one or more computer servers, which can enable distributed computing, such as cloud computing. For example, one or more computer servers may enable cloud computing over the network 130 (“the cloud”) to perform various aspects of analysis, calculation, and generation of the present disclosure, such as, for example, obtaining sequencing information of a sample, identifying organisms or microbes in a population, identifying metabolic pathways or reactions associated with organisms or microbes in a population, identifying presence of metabolic pathways, as indicated by identifying a nucleic acid marker that encodes a component of the metabolic pathway in a genome of the organism, and determining abundance of metabolic pathways. Such cloud computing may be provided by cloud computing platforms such as, for example, Amazon Web Services (AWS), Microsoft Azure, Google Cloud Platform, and IBM cloud. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1010. Examples of user interfaces (UIs) include, without limitation, a graphical user interface (GUI) and web-based user interface. For example, the computer system can include a web-based dashboard (e.g., a GUI) configured to display, for example, obtained sequencing information of a sample, identified organisms or microbes in a population, identified metabolic pathways or reactions associated with organisms or microbes in a population, identified presence of metabolic pathways, as indicated by identifying a nucleic acid marker that encodes a component of the metabolic pathway in a genome of the organism, and determined abundance of metabolic pathways.

Methods and systems of the present disclosure can be implemented by way of one or more algorithms. An algorithm can be implemented by way of software upon execution by the central processing unit 1005. The algorithm can, for example, obtain sequencing information of a sample, identifying organisms or microbes in a population, identify metabolic pathways or reactions associated with organisms or microbes in a population, identify presence of metabolic pathways, as indicated by identifying a nucleic acid marker that encodes a component of the metabolic pathway in a genome of the organism, and determine abundance of metabolic pathways.

Although the description has been described with respect to particular embodiments thereof, these particular embodiments are merely illustrative, and not restrictive. Concepts illustrated in the examples may be applied to other examples and implementations.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

These examples are provided for illustrative purposes only and not to limit the scope of the claims provided herein.

### Example 1: Microbiome Metabolic Pathway Prediction to Predict Presence or Absence of a Pathway

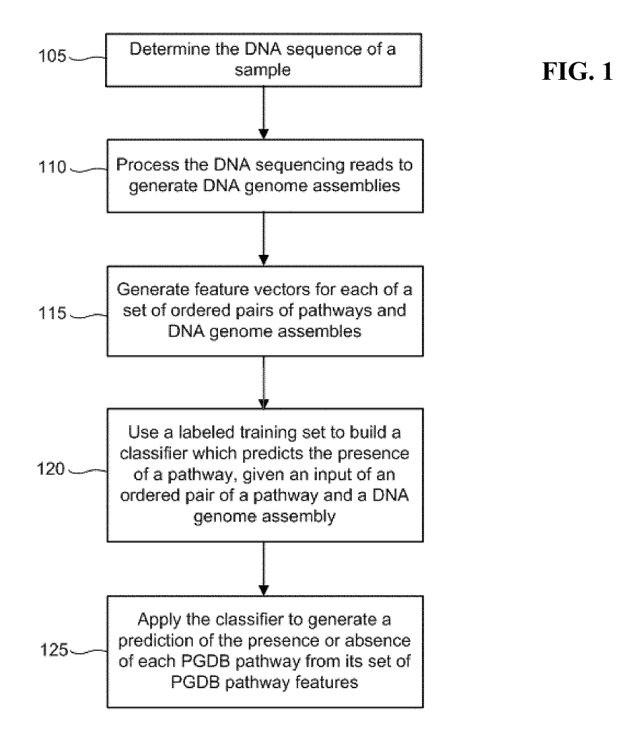

The following method can be performed to predict whether a pathway is present or absent, given a DNA sequence typically representing either an organism's genome or an environment's metagenome, exemplified in FIG. 1.

First, the DNA sequence of the sample is determined (e.g., 105). Second, the DNA sequencing reads are processed to generate DNA contigs or genome assemblies (e.g., 110). Third, feature vectors for each of a set of ordered pairs of a MetaCyc Pathway and a DNA genome assembly (e.g., genes encoded, putative taxa, reactome, etc.) are generated (e.g., 115). Fourth, a labeled training set is used to build a classifier which predicts the presence of a pathway, given an input of an ordered pair of a pathway and a DNA genome assembly (e.g., 120). A metacyc pathway refers to a pathway in metacyc, and hence not associated with an organism. A PGDB pathway refers to a pathway associated with an organism. For example, ‘12DICHLORETHDEG-PWY’ is a metacyc pathway and ‘12DICHLORETHDEG-PWY in yeast’ in a PGDB pathway. Fifth, the microbiome metabolis pathway prediction algorithm applies the classifier to generate a prediction of the status (absent or present) of each PGDB pathway from its set of PGDB pathway features (e.g., 125).

A training set is used, in which each PGDB pathway is associated with its features and a known status (i.e., if the pathway is present or absent is known). The level of confidence in the ‘known status’ differs for each PGDB pathway depending on its tier and level of curation.

Organisms from different ‘tiers’ corresponding to different levels of pathway curation/confidence are used in the absence/presence determination.

Tier 1 indicates a very high confidence level in the PGDB pathway status,

Tier 2 indicates a high confidence level in the PGDB pathway status,

Tier 3 indicates a lower confidence level in the PGDB pathway status.

There are 2391 PGDB pathways per organism. The number of pathways per tier is: Tier 1=6 (excluding MetaCyc), Tier 2=48, Tier 3=9318.

For each PGDB pathway, the true status (e.g., presence or absence) is not known. But, as the ground truth, a mixture of computational predictions (pathologic algorithm) and manual curation is used. About 91% of the PGDB pathways are present in the training set. Therefore, predicting that all the PGDB pathways are absent gives an prediction accuracy of about 91%. Comparing the proportion of PGDB pathways that are absent and present, 90.1% of the PGDB pathways are absent, and 9.9% of the PGDB pathways are present.

Features may be extracted from each of a set of ordered pairs of a pathway and a genome assembly, including some or all of the MetaCyc Pathway features and/or PGDB Pathway features, as described below.

The following features may be collectively referred to as MetaCyc Pathway features:

[1] “Common Name”: a string value which indicates the common name of the MetaCyc pathway

[2] “num-reactions”: an integer value which quantifies the number of reactions present in the MetaCyc pathway (the number of MetaCyc pathway reactions which have assigned enzymes).

[3] “num-key-reactions”: an integer value which quantifies the number of key reactions present in the MetaCyc pathway (the number of key MetaCyc pathway reactions which have assigned enzymes).

[4] “is-subpathway”: a binary or Boolean value which indicates whether or not a pathway is a subpathway.

[5] “biosynthesis-pathway”: a binary or Boolean value which indicates whether or not a pathway is a biosynthesis pathway (a pathway which has biosynthesis enzymes assigned to it).

[6] “degradation-pathway”: a binary or Boolean value which indicates whether or not a pathway is a degradation pathway (a pathway which has degradation enzymes assigned to it).

[7] “detoxification-pathway”: a binary or Boolean value which indicates whether or not a pathway is a detoxification pathway (a pathway which has detoxification enzymes assigned to it).

[8] “pwy-uniq-norm”: a real value which quantifies a normalized weighted sum, which is calculated by the weighted sum of inverse reaction frequencies, normalized by the number of reactions in the pathway. The frequency of a reaction is the number of distinct pathways that it occurs in.

[9] “pwy-uniq-nonorm”: a real value which quantifies an un-normalized weighted sum, which is calculated by the weighted sum of inverse reaction frequencies (the numerator portion of “pwy-uniq-norm”).

[10] “num-enz-rxns”: an integer value which quantifies the number of reactions in the MetaCyc pathway that are annotated as having an associated enzyme. Any MetaCyc pathway having zero such reactions should be excluded from pathway prediction.

[11] “glycan-pathway”: a binary or Boolean value which indicates whether or not the pathway is of type “Glycan-Pathways”. These pathways have a complex representation and may be excluded from consideration until a better understanding of how to handle this special type is obtained.

The following features may be collectively referred to as PGDB Pathway features:

[12] “is-present”: a binary or Boolean value which indicates whether or not the pathway is present in the PGDB. This value may not specify how the pathway was created. In the case of Tier 1 and Tier 2 PGDBs, it could have been created automatically by PathoLogic, or it could have been manually curated.

[13] “num-reactions-present”: an integer value which quantifies the number of reactions present in the PGDB pathway (the number of PGDB pathway reactions which have assigned enzymes).

[14] “all-key-reactions-are-present”: a binary or Boolean value which indicates whether or not all key reactions in a PGDB pathway are present (whether or not all of the key reactions curated in the MetaCyc pathway have assigned enzymes in the PGDB pathway).

[15] “num-key-reactions-present”: an integer value which quantifies the number of key reactions curated in the MetaCyc pathway that are present in the PGDB pathway (the number of key PGDB pathway reactions which have assigned enzymes).

[16] “fraction-key-reactions-present”: a fraction or percentage value which quantifies the proportion of key reactions present in the PGDB pathway (the fraction or percentage of key reactions curated in the MetaCyc pathway that have assigned enzymes in the PGDB pathway). This may also be defined as num-key-reactions-present or num-key-reactions.

[17] “taxonomic-range-includes-target-alt”: as defined in the Dale supplemental

[18] “fraction-reactions-with-enzymes”: a fraction or percentage value which quantifies the fraction of PGDB pathway reactions that have assigned enzymes. This may also be defined as num-reactions-present or num-reactions.

[19] “all-rxns-are-present”: a binary or Boolean value which indicates whether or not every PGDB pathway reaction has assigned enzymes (whether or not fraction-reactions-with-enzymes is equal to 1).

[20] “has-enzymes”: a binary or Boolean value which indicates whether or not at least some of the PGDB pathway reactions have assigned enzymes (whether or not fraction-reactions-with-enzymes is greater than 0).

[21] “has-unique-enzymes”: a binary or Boolean value which indicates whether or not the MetaCyc pathway's unique reactions have assigned enzymes in the PGDB pathway. A MetaCyc pathway may have a unique reaction if that reaction is only present in that MetaCyc pathway, and no other.

[22] “num-unique-enzymes”: an integer value which quantifies the number of unique MetaCyc pathway reactions that have assigned enzymes in the PGDB pathway.

[23] “fraction-unique-enzymes-present”: a fraction or percentage value which indicates the proportion of unique MetaCyc pathway reactions that have assigned enzymes in the PGDB pathway.

[24] “enzyme-info-content-norm”: a real value which quantifies the normalized weighted sum of the PGDB enzymes that catalyze the reactions of a PGDB pathway. Each PGDB enzyme is weighted as the inverse of the frequency at which the PGDB enzyme is assigned to PGDB pathways. Therefore, an enzyme that is present and is only found in the current PGDB pathway has a weight of one. As another example, an enzyme which is assigned to a reaction in the current PGDB pathway, and also assigned to nine additional PGDB pathways, will have a weight of 1/10. The weighted sum is normalized by the number of enzymes that are present and associated with the PGDB pathway.

[25] “enzyme-info-content-no-norm”: a real value which quantifies the un-normalized weighted sum of the PGDB enzymes that catalyze the reactions of a PGDB pathway.

[26] “reaction-info-content-norm”: similar to enzyme-info-content-norm, a real value which quantifies the normalized weighted sum of reactions with assigned enzymes, wherein the weighting is the frequency of the reaction among MetaCyc pathways. The normalization “denominator” is the same as “num-reactions.”

[27] “reaction-info-content-no-norm”: a real value which quantifies the un-normalized weighted sum of reactions with assigned enzymes.

[28] “not-mostly-absent”: a binary or Boolean value which indicates whether or not a pathway is mostly absent. The concept of “mostly absent” is described, for example, by Karp et al. [“The Pathway Tools Pathway Prediction Algorithm,” Karp, Peter D., Latendresse, Mario, and Caspi, Ron, Stand Geonomic Sci., 2011, 5:3], which is hereby incorporated by reference in its entirety.

[29] “pathologic-pred”: an approximation of the PathoLogic prediction (as described by Karp et al.) which uses a logic expression involving a few features, such as: not-mostly-absent, all-key-reactions-are-present, has-unique-enzymes, taxonomic-range-includes-target-alt, num-rxns, and num-rxns-present.

[30] “num-pathway-holes”: an integer value which quantifies the number of pathway holes in the PGDB pathway. This can also be defined as num-reactions or num-reactions-present.

[31] “manually-curated”: a binary or Boolean value which indicates whether or not, based on PGDB pathway evidence codes, the PGDB pathway was manually curated.

[32] “rxn-set-difference”: a binary or Boolean value which indicates whether or not the PGDB pathway reaction set is different from the MetaCyc pathway reaction set. This value indicates that the PGDB pathway is out-of-date from the most recent version of MetaCyc. Such pathways are excluded from consideration.

[33] “manually-curated-parts”: a binary or Boolean value which indicates whether or not the pathway has any enzymes, reactions, or enzymatic reactions with evidence of manual curation.

[34] “partial-pwy-evidence”: a real value which quantifies a combination of “rxn-info-content-norm” and “manually-curated-parts.” If a reaction (or its enzrxn, or its enzyme) has any evidence of manual curation, a weighted sum is calculated, wherein each weight is the frequency at which the reaction appears in MetaCyc pathways, and then normalized by the number of eligible reactions (similar to “rxn-info-content-norm,” the normalization “denominator” is the same as “num-reactions”).

A probability that a PGDB pathway is present or absent may be modeled using a logistic regression (logit) or a random forest (rf).

Let Y be a binary random variable indicating whether a given PGDB pathway is present. Let (X₁, . . . , Xₙ) be a set of variable with observed values (x₁, . . . , xₙ). The training data set is denoted by D.

To model a logistic regression, the R function glm was used. The logit of the probability that a PGDB pathway is present or not is modeled as a linear function of the variables.

Let

π=P(Y=1|X₁=x₁, . . . ,Xₙ=xₙ).

Assume that

$$log ( \frac{π}{1 - π} ) = β x = {β}_{0} + {β}_{1} {x}_{1} + … + {β}_{n} {x}_{n} .$$

The parameter of interest is β. Once β is estimated, π is estimated by:

$$π = P ( Y = 1 \vert X ) = \frac{1}{1 + {e}^{- β x}} .$$

A threshold T is estimated to infer whether a pathway is present or not.

$$Y = \{ \begin{matrix}1 & if \hat{π} > T , \\ 0 & otherwise\end{matrix} .$$

Set T=0.5 when computing the performance measures. R function glm with binomial family was used.

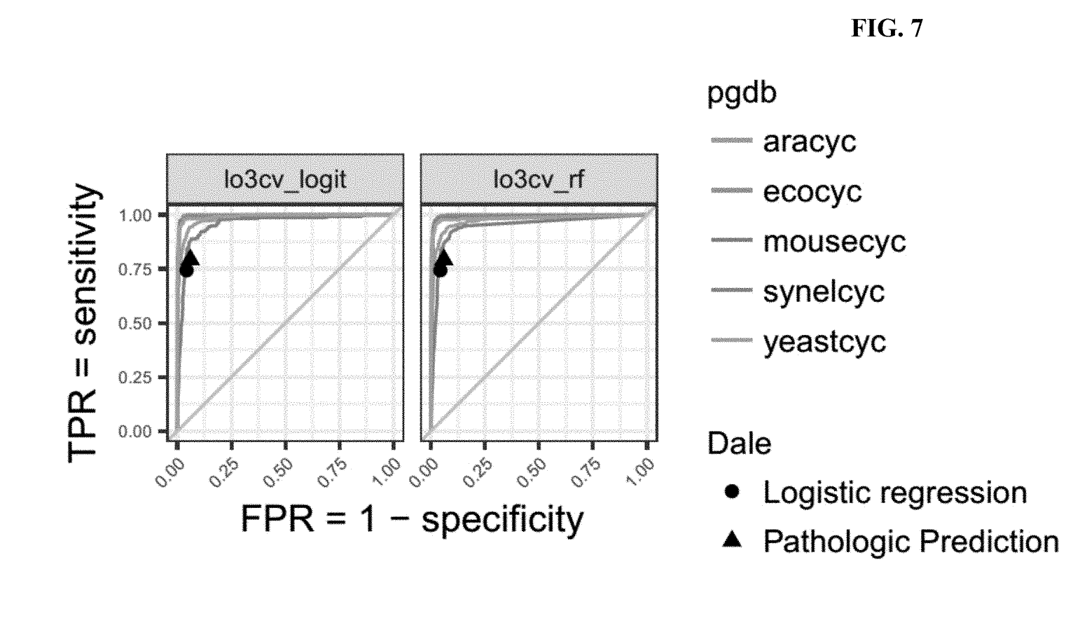

Results of analysis using a logistic regression are provided in FIG. 7. To model a random forest (rf), the R function randomForest was used. A random forest (rf) can be described as follows. “A decision tree predictor consists of a tree data structure where each internal node of the tree represents a test of one of the input features used for prediction, for example, testing whether the value of a Boolean feature is true, or whether the value of a numeric feature is less than a threshold value stored at the node. For each possible outcome of the test, there is a corresponding subtree. Each leaf node in the tree stores the numbers of present and absent training instances that satisfy all the tests between the root node and that leaf node. The decision tree prediction algorithm involves traversing the tree structure by applying the node tests to the instance being classified, starting with the test at the root of the tree, and continuing on to the subtree selected by the test. When a leaf node is reached, the counts of training examples at the leaf are used to make either a Boolean prediction (true if the majority of training instances at that node are present, false otherwise) or a numeric prediction (estimating the probability that the instance is present by the fraction of training instances at the node that are present),” as described by Dale et al. [“Machine learning methods for metabolic pathway prediction,” Dale, Joseph M., Popescu, Liviu, and Karp, Peter D., BMC Bioinformatics, 2010, 11:15], which is hereby incorporated by reference in its entirety.

The random forest method is used, in which the training dataset is resampled with replacement (e.g., given a training dataset D, a new dataset D′, of the same size as D, is constructed by selecting instances (sampling) from D at random with replacement) and mtry variables are also randomly selected. Next, a decision tree predictor is trained on the resampled dataset. This process (re-sampling and training) is repeated ntree times, and the resulting set of ntree predictors is taken as an ensemble predictor.

The R function randomForest from package randomForest was used, in which the number of sampled variables was mtry=3 and the number of trees built was ntree=500.

Estimation of prediction errors in the pathway prediction can be performed using Monte-Carlo cross-validation (MCCV), leave one organism out cross-validation (LO3CV), or a combination thereof.

Monte-Carlo cross-validation (MCCV) estimates prediction performance when the trained predictor is trained and evaluated on the same set of organisms. Then, the MCCV estimates the prediction performance of the trained predictor in predicting the status of a new PGDB pathway from an organism, when this same organism was present in the training set.

The MCCV procedure is performed as follows:

1. Sample at random, without replacement, 80% of the PGDB pathways to obtain a sampled set of PGDB pathways.

2. Fit the model (e.g., logistic regression or random forest) on the sampled set of PGDB pathways, by performing the random forest training on the sampled set of PGDB pathways to generate a predictor.

3. Predict on the remaining PGDB pathways, by using the predictor to make pathway predictions for the remaining set of PGDB pathways (e.g., the remaining 20% of the PGDB pathways which were not randomly sampled). Measure the prediction performance (e.g., accuracy) of the set of such predictions.

4. Repeat steps 1 to 3 for a total of n (n=20) times.

5. Average prediction performances over the n replicates of models.

For random forest, there may be no need to perform cross-validation to obtain an unbiased estimate of the test set error, since such an estimate is obtained internally using the out-of-bag (OOB) error. To be consistent and compare random forest and logistic regression results, results for MCCV for random forest are shown. OOB and MCCV error estimates were similar.

Leave one organism out cross-validation (LO3CV) evaluates prediction performance when the predictor is trained and evaluated on a set of different organisms. Then, the LO3CV estimates the prediction performance of the trained predictor in predicting the status of PGDB pathways from an organism was not trained on (e.g., not included in the training dataset).

The LO3CV procedure is performed as follows:

1. Fit the model (e.g., logistic regression or random forest) on a set of N−1 organisms, where N is the total number of organisms (e.g., by leaving one organism out of the set).

2. Predict on the remaining organism (left out of the set), by using the predictor to make pathway predictions for the remaining organism. Measure the prediction performance (e.g., accuracy) of the set of such predictions.

3. Repeat steps 1 and 2 for a total of N times (cycling through each of the set of N organisms).

4. Average prediction performances over the models corresponding to the N organisms.

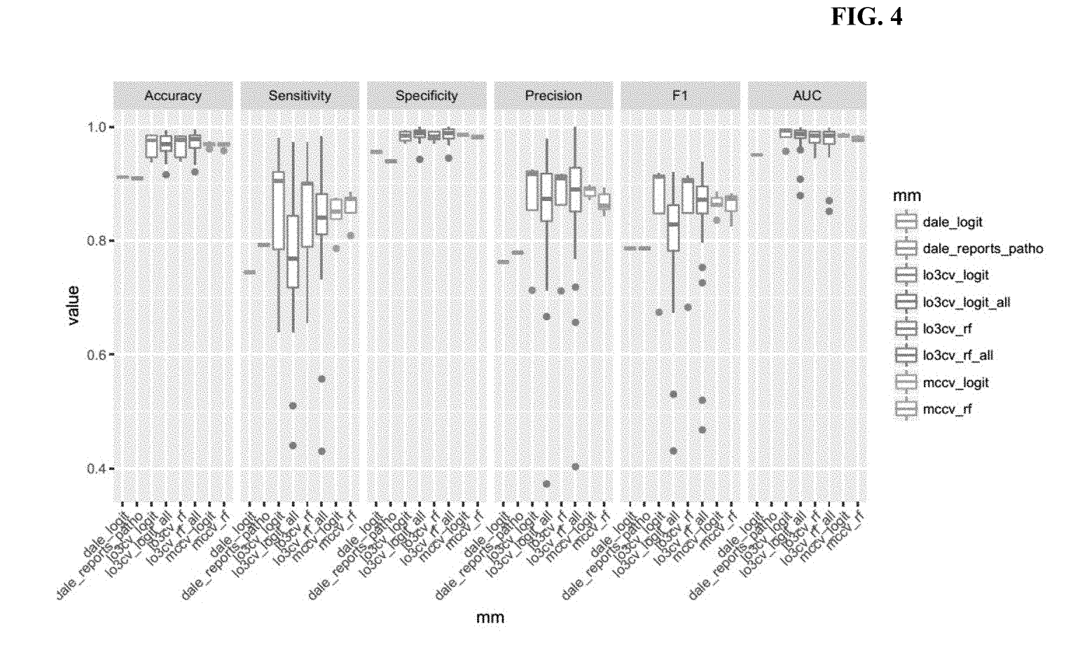

FIG. 4 exemplifies and compares performance of a method of pathway prediction of the present disclosure with other methods of pathway prediction. Performance measures indicated by dale_logit and dale_reports_patho correspond to methods of pathway prediction as disclosed by Dale et al., using logistic regression and PathoLogic approaches, respectively. Performance measures indicated by lo3cv_logit and lo3cv_logit_all correspond to methods of pathway prediction of the present disclosure, using a logistic regression and leave one organism out cross-validation (LO3CV). Performance measures indicated by lo3cv_rf and lo3cv_rf_all correspond to methods of pathway prediction of the present disclosure, using a random forest and leave one organism out cross-validation (LO3CV). Performance measures indicated by mccv_logit correspond to methods of pathway prediction of the present disclosure, using a logistic regression and Monte Carlo cross-validation (MCCV). Performance measures indicated by mccv_rf correspond to methods of pathway prediction of the present disclosure, using a random forest and Monte Carlo cross-validation (MCCV).

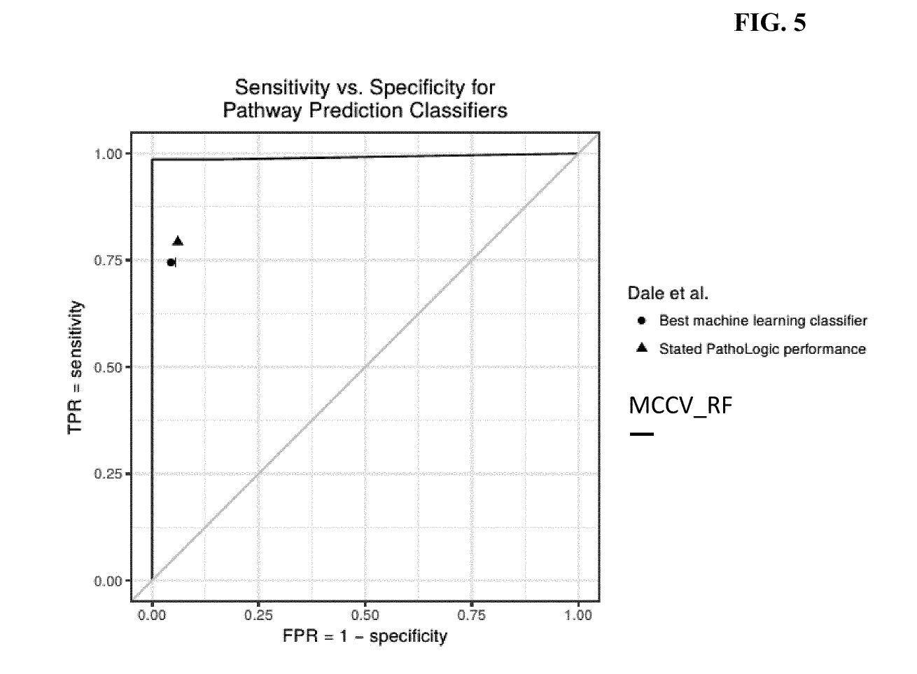

FIG. 5 exemplifies the improvement in sensitivity and specificity values of an approach of the present disclosure (using a random forest model, denoted by a solid line, MCCV_RF) versus current methods of pathway prediction disclosed by Dale et al. (a machine learning classifier, denoted by a point with a circle icon; and a stated PathoLogic approach, denoted by a point with a triangle icon). The Monte Carlo cross-validation (MCCV) method was used for all three methods of pathway prediction.

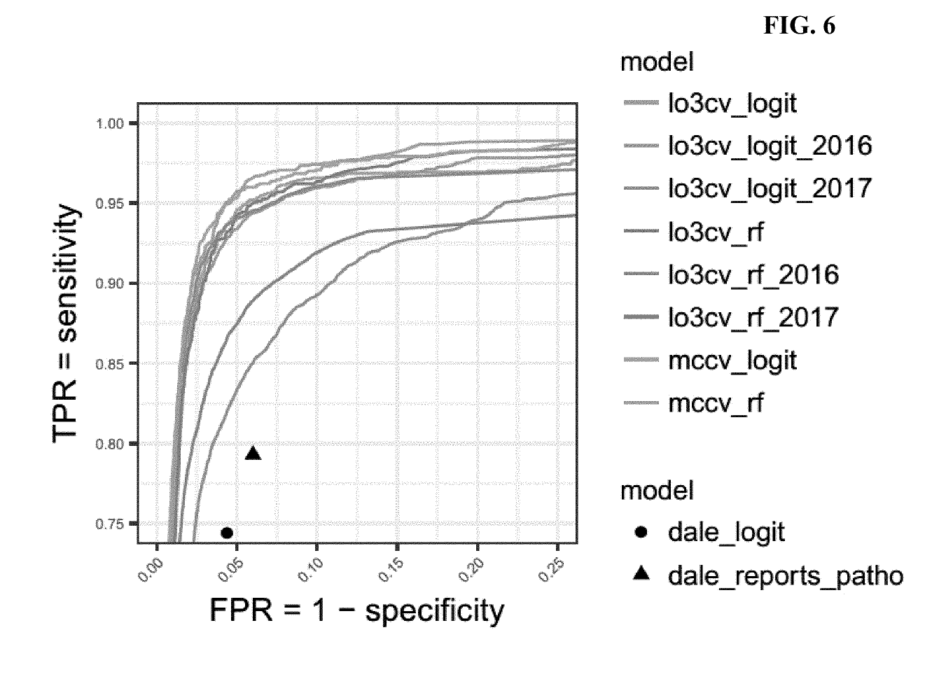

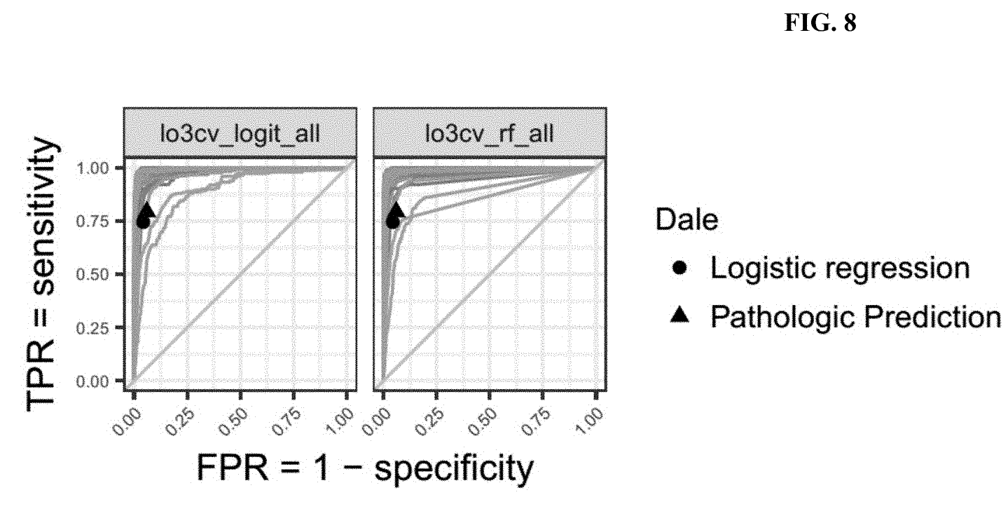

Comparing the MCCV and LO3CV approaches, leave one organism out cross validation (LO3CV) performed on average equally or better than Monte Carlo cross validation (MCCV), but with greater variance. In particular, variance is high in this dataset because the number of organisms is small. Leave one organism out cross validation variance is reduced when 28 organisms from 3 different tiers were used. Results of the leave one organism out cross validation (LO3CV) and Monte Carlo cross validation (MCCV) approaches are provided in FIGS. 6, 7, and 8.

When using logistic regression and random forest to predict the PGDB pathways status, an assumption may be made that the PGDB pathways are independent. This assumption may not be optimal in certain circumstances. In particular, some reactions are shared by different pathways. Therefore, not accounting for the dependence between pathways may result in suboptimal prediction performance. A prediction model to jointly predict the presence or absence of sets of PGDB pathways may be developed. Relaxing the independence assumption may give a more realistic understanding of the functional potential of a metagenomic sample and/or more optimal prediction performance compared to when logistic regression or random forest is used.

### Example 2: Microbiome Metabolic Pathway Prediction

The observed enzyme abundances are formally modeled as a linear mixture model of the organisms present. The mixture modeling method first determines which taxa are present in a sample using MetaPhlAn2. The method then maps the observed taxa to the 7,600 organism Pathway/Genome Databases (PGDBs) present in the BioCyc collection, with the individual PGDB pathways computationally predicted using the Pathway Tools PathoLogic module (which has an accuracy of 91%). A system of linear equations of the form Ax≈b is obtained, where x is a mixing vector (i.e., the non-negative abundance of each PGDB or enzyme grouping), and b is a set of observed enzymes. This constrained, overdetermined regression problem is solved using weighted, regularized, non-negative least squares (NNLS). The NNLS problem is formulated using elastic net regularization regression routines used in machine learning (using the glmnet R package).

The method is validated using the Human Microbiome Project Mock Community resource, which provides NGS reads obtained from in vitro mixtures of fully-sequenced microbes in fixed ratios. Next, extensive in silico simulation of NGS reads with errors drawn from complex communities of hundreds of strains is performed, for a more thorough validation of the method.

The performance of the method in terms of microbiome metabolic pathway prediction is compared to other methods such as PathoLogic, bagged HC-BIC method, and MinPath. Table 1 below shows the improved prediction accuracy of the methods of the present disclosure (“Present”) over other methods.

TABLE 1

| Prediction | | | | Bagged | |
|---|---|---|---|---|---|
| Performance | Present | Present | PathoLogic | HC-BIC | MinPath |
| Metric | Mean | Median | Mean | Mean | Mean |
| Accuracy | 0.998 | 0.998 | 0.91 | 0.912 | 0.886 |
| Sensitivity | 0.986 | 0.982 | 0.793 | 0.744 | 0.993 |
| Specificity | 1.000 | 1.000 | 0.94 | 0.956 | 0.871 |

### Example 3: Use of Time-Series Analysis for Metabolic Pathway Prediction

A time-series algorithm is integrated into microbiome metabolic pathway prediction methods and is used to follow the changes in the microbiome and predict metabolic pathway changes in subjects being treated with compositions comprising specific microbes as part of therapeutic intervention. Biological samples are collected from patients undergoing therapy 1) before therapy starts and 2) every alternate day for 2 weeks. The samples are processed and analyzed using methods of the present disclosure to predict the microbiome metabolic pathway. The time-series algorithm also follows changes over time corresponding to samples collected. The results are combined to predict the efficacy of the microbial composition being administered to the subject and the changes in their microbiome over the course of the treatment.

### Example 4: Use of Integrated Platform with the Pathway Prediction, Machine Learning (e.g., Deep Learning), and Time-Series to Predict Pathways Related to Butyrate Production

Biological samples from subjects are collected and analyzed using the integrated platform with the metabolic pathway prediction, machine learning (e.g., deep learning), and time-series algorithms. In particular, pathways related to butyrate production are analyzed to determine 1) the microbiome pathways status in the subject, 2) whether the subject needs to be administered a composition comprising butyrate-producing microbes, 3) whether and how the pathway changes over time with different combinations of the compositions administered, and 4) whether the subject will respond to the specific therapeutic intervention. Based on the analysis, the microbial therapy regimen is customized to the needs of the subject.

### Example 5: Use of Gut Microbiome Metabolic Map to Select Responders to Increasing Butyrate Production

To determine if a subject will respond to a particular composition of microbes, their metabolic map is determined. The biological samples collected are processed and the metabolic map is generated from the collected biological samples using a combination of 16S sequencing, metagenomic sequencing and metabolite profiling. Based on the microbiome metabolic map determined, markers to predict responders to butyrate production are identified. Prediction markers and the corresponding treatment based on the metabolic map of the subject are shown in Table 2 below:

TABLE 2

| | Strains producing | |
|---|---|---|
| | carbohydrates and | |
| | sugars that | |
| Butyrate levels in | feed butyrate | |
| sample | producing strains | Marker - Treatment |
| Normal | Normal | Healthy |
| Normal | Reduced | Administer fiber |
| Low | Reduced | Synbiotic - Administer fiber + |
| | | Butyrate producing strains |
| Low | Normal | Probiotic - Administer butyrate |
| | | producing strains |

1. A method of determining an abundance of a metabolic pathway from a sample comprising a population of a plurality of different organisms, the method comprising:
(a) obtaining sequencing information from nucleic acid molecules in the population;
(b) determining a presence of a nucleic acid marker that encodes a component of the metabolic pathway in a genome of each of one or more organisms in the plurality of different organisms in the population, comprising:
(i) identifying an organism in the population based on the sequencing information, and
(ii) determining a presence of the nucleic acid marker from the organism in an identified set of reactions, wherein the nucleic acid marker encodes the component of the metabolic pathway in the genome of the organism; and
(c) determining the abundance of the nucleic acid marker from the plurality of different organisms in the population, thereby determining an abundance of the metabolic pathway in the population.

2. (canceled)

3. The method of claim 1, wherein the abundance comprises a relative or normal abundance.

4. (canceled)

5. The method of claim 1, wherein the nucleic acid marker encodes an enzyme in the metabolic pathway.

6. The method of claim 5, wherein (c) comprises determining the abundance of the metabolic pathway based at least in part on an abundance of the nucleic acid marker that comprises a sequence encoding an enzyme in the metabolic pathway.

7. The method of claim 1, wherein the metabolic pathway comprises a distributed metabolic pathway.

8. The method of claim 7, wherein the distributed metabolic pathway is catalyzed by a plurality of organisms.

9. (canceled)

10. (canceled)

11. The method of claim 1, wherein determining the presence of the metabolic pathway comprises querying a database based on the organism identified in (i).

12. The method of claim 1, wherein the metabolic pathway is identified using a model trained with metabolic pathway data that is tiered, each tier of metabolic pathway data corresponding to a different discrete range of confidence level in the metabolic pathway data.

13. The method of claim 1, further comprising generating one or more feature vectors for the organism and the nucleic acid marker from the organism.

14. The method of claim 13, wherein the one or more feature vectors are selected from the group consisting of: reaction-info-content-norm, fraction-reactions-with-enzymes, taxonomic-range-includes-target-alt, enzyme-info-content-norm, all-rxns-are-present, num-pathway-holes, not-mostly-absent, rxn-set-difference, manually-curated-parts, partial-pwy-evidence, manually-curated, glycan-pathway, and any combination thereof.

15. The method of claim 13, further comprising using the one or more feature vectors to determine the abundance of the nucleic acid marker from the organism, wherein the one or more feature vectors are indicative of presence of the metabolic pathway.

16. The method of claim 1, wherein determining the presence of the nucleic acid marker from the organism further comprises using a machine learning algorithm trained with a set of metabolic pathways that are known to be present or absent in the one or more organisms in the plurality.

17. The method of claim 16, wherein the machine learning algorithm is configured to determine the abundance nucleic acid marker of a distributed metabolic pathway.

18. The method of claim 17, wherein the distributed metabolic pathway is catalyzed at least in part by two or more microbes in the population.

19. The method of claim 18, wherein the distributed metabolic pathway has transporters for intermediate metabolites catalyzed by the microbe.

20. The method of claim 16, wherein the machine learning algorithm comprises a random forest.

21. (canceled)

22. (canceled)

23. (canceled)

24. The method of claim 1, wherein the metabolic pathway is associated with production of short-chain fatty acids (SCFAs).

25. The method of claim 1, wherein obtaining the sequencing information comprises sequencing a nucleic acid sequence of a ribosomal RNA operon in the sample.

26. The method of claim 1, wherein the presence of the nucleic acid marker is identified with a mean or median accuracy of at least about 92%.

27.-30. (canceled)

31. The method of claim 26, wherein the presence of the nucleic acid marker is identified with a mean or median sensitivity of at least about 80%.

32.-70. (canceled)

71. The method of claim 1, further comprising identifying the set of reactions for the organism of (i).
